(19) United States
(12) Patent Application Publication
(10) Pub. No.: US 2014/0066488 A1
(43) Pub. Date: Mar. 6, 2014

(54) Inhibitors of Memapsin 2 Cleavage for the Treatment of Alzheimer's Disease

(71) Applicants: Purdue Research Foundation, West Lafayette, IN (US); Oklahoma Medical Research Foundation, Oklahoma City, OK (US)

(72) Inventors: Jordan Tang, Edmond, OK (US); Venkateswararao Kalapala, West Lafayette, IN (US); Arun K. Ghosh, West Lafayette, IN (US)

(73) Assignees: Purdue Research Foundation, West Lafayette, IN (US); Oklahoma Medical Research Foundation, Oklahoma City, OK (US)

(21) Appl. No.: 14/012,293

(22) Filed: Aug. 28, 2013

Related U.S. Application Data
(60) Provisional application No. 61/695,148, filed on Aug. 30, 2012.

(51) Int. Cl. C07D 513/06; A61K 45/06; A61K 31/554; C07C 311/07; A61K 31/18

(52) U.S. Cl. CPC C07D 513/06; C07C 311/07; A61K 31/18; A61K 31/554; A61K 45/06
USPC 514/411; 564/99; 540/545; 514/605; 435/184

(57) ABSTRACT

Proteases such as memapsin 2 are important enzymes, playing roles in a variety of diseases including Alzheimer's Disease. The inventors have developed inhibitors of memapsin 2 and methods of use therefore in the treatment of disease.

This application claims benefit of priority to U.S. Provisional Application Ser. No. 61/695,148, filed Aug. 30, 2012, the entire contents of which are hereby incorporated by reference.

### STATEMENT OF FEDERAL FUNDING

This invention was made with government support under grant no. AG-18933 awarded by the National Institutes of Health. The government has certain rights in the invention.

### BACKGROUND OF THE INVENTION

I. Field of the Invention

The present invention relates generally to the fields of enzymology and biochemistry. More particularly, it concerns the inhibition of the memapsin 2 (BACE 1) protease and the treatment of memapsin 2-related diseases, including Alzheimer's Disease.

II. Description of Related Art

Alzheimer's disease (AD) is a progressive, degenerative brain disorder with no effective treatment to date, and the development of new drugs is an urgent priority in medicine (Goedert and Spillantini, 2006). The hallmark of AD is the formation of neutritic plaques containing 40/42 residue amyloid-β (Aβ) peptides and neurofibrillary tangles in the brain (Selkoe and Schenk, 2003) β-Secretase (memapsin 2, β-site APP cleaving enzyme 1 (BACE 1)) is one of two proteases that cleaves β-amyloid precursor protein (APP) and generates Aβ and its aggregation product (Citron, 2010). There is considerable evidence that excess Aβ leads to brain inflammation, neuronal death, and AD (Billings et al., 2005). Consequently, β-secretase has become a major therapeutic target for drug development (Ghosh et al., 2012 and Tang et al., 2010). Since the inventors' design of initial transition-state inhibitor (1, FIG. 1) and subsequent determination of inhibitor-bound memapsin 2 X-ray structure, nearly a decade ago, steady progress has been made toward the evolution of small molecule potent and brain-penetrable inhibitor drugs (Ghosh et al., 2000 and Hong et al., 2000).

Recently, the inventors have shown that administration of β-secretase inhibitor 2 rescued cognitive decline in transgenic AD mice, validating β-secretase as an important drug design target (Ghosh et al., 2008 and Chang et al., 2011). However, the development of clinical β-secretase inhibitor drug is faced with numerous formidable challenges, including lack of selectivity against other physiologically important aspartic acid proteases and issues of poor pharmacological profiles including blood-brain penetration (Ghosh et al., 2000 and Hong et al., 2000). In continuing work toward the design of small molecule potent and selective inhibitors, the inventors have been particularly interested in developing tools for selectivity against relevant physiologically important aspartic acid proteases, especially cathepsin D (CD) and β-site APP cleaving enzyme 2 (BACE 2). BACE 2 has specificity similarity to BACE 1, and this is known to have important physiological functions (Turner et al., 2002). CD plays a key role in important biological functions like protein catabolism (Diment et al., 1988). The abundance of CD in various cells, especially in central nervous system tissue cells, is very high. Furthermore, CD gene knockout studies in mice showed marked phenotypic response including high mortality rate (Koike et al., 2000 and Saftig et al., 1995). Therefore, the selective inhibition of β-secretase over CD and BACE 2 is very critical to reduce toxicity and other side effects of β-secretase inhibitor drugs.

As described by the inventors previously, the X-ray crystal structure of inhibitor 1-bound β-secretase showed an interesting hydrogen bonding between the P2′-carbonyl and the hydroxyl of Tyr-198, forming a rare kink at the P2′ site (Hong et al., 2000). They have exploited this interaction in the design and synthesis of very potent and highly selective β-secretase inhibitors such as 3 by incorporating hydroxyethylene isosteres (Ghosh et al., 2006). However, the cellular β-secretase inhibitory activity of this class of inhibitors was only in the micromolar range. Enzyme inhibitors containing reduced amide isostere have been reported; however, they exhibited only marginal selectivity against memapsin 1 (BACE 2) (Tang et al., 2010; Iserloh and Cumming, 2010 and Coburn et al., 2006).

### SUMMARY OF THE INVENTION

Thus, in accordance with the present invention, there is provided a method of inhibiting memapsin 2 activity comprising contacting a memapsin 2 enzyme with a compound having a formula selected from:

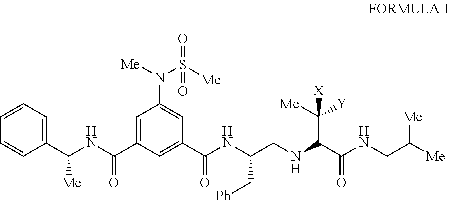

wherein X and Y are H or OH; or

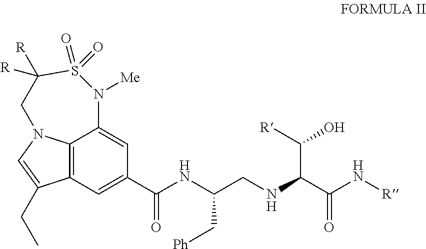

wherein each R, R′ and R′ are independently selected from C_{<8} alkyl, C_{<8} substituted alkyl, C_{<8} heterocycloalkyl, C_{<8} alkoxyalkyl, C_{<9} alkylamino, C_{<12} aryl, C_{<12} arylalkyl,
or a pharmaceutically acceptable salt or tautomer of any of the above formulas. The compound may have formula I, wherein X is H and Y is OH. Alternatively, the compound may have formula I, wherein X is H and Y is H. The compound may have formula II, wherein (i) R is H, R′ is —CH₃, and R″ is isobutyl, or (ii) wherein R is H, R′ is n-propyl, and R″ is isobutyl, or (iii) wherein R is H, R′ is isopropyl, and R″ is isobutyl, or (iv) wherein each R together form —CH₂—CH₂—, R′ is —CH₃, and R″ is isobutyl, or (v) wherein each R together form —CH₂—CH₂—, R′ is n-propyl, and R″ is isobutyl, or (vi) wherein each R together form —CH₂—CH₂—, R′ is isopropyl, and R″ is isobutyl, or (vii) wherein R′ is isopropyl.

In another embodiment, there is provided a method of treating a mammalian subject with Alzheimer's Disease comprising administering to said subject a compound having a formula selected from:

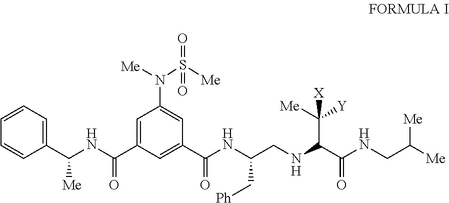

wherein X and Y are H or OH; or

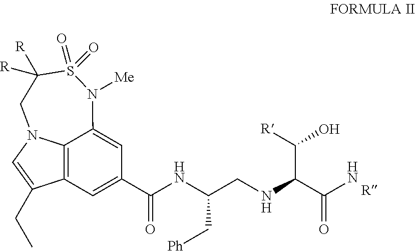

wherein each R, R′ and R′ are independently selected from C_{<8} alkyl, C_{<8} substituted alkyl, C_{<8} heterocycloalkyl, C_{<8} alkoxyalkyl, C_{<9} alkylamino, C_{<12} aryl, C_{<12} arylalkyl,
or a pharmaceutically acceptable salt or tautomer of any of the above formulas. The compound may have formula I, wherein X is H and Y is OH. Alternatively, the compound may have formula I, wherein X is H and Y is H. The compound may have formula II, wherein (i) R is H, R′ is —CH₃, and R″ is isobutyl, or (ii) wherein R is H, R′ is n-propyl, and R″ is isobutyl, or (iii) wherein R is H, R′ is isopropyl, and R″ is isobutyl, or (iv) wherein each R together form —CH₂—CH₂—, R′ is —CH₃, and R″ is isobutyl, or (v) wherein each R together form —CH₂—CH₂—, R′ is n-propyl, and R″ is isobutyl, or (vi) wherein each R together form —CH₂—CH₂—, R′ is isopropyl, and R″ is isobutyl, or (vii) wherein R′ is isopropyl.

The subject may be further treated with at least a second Alzheimer's Disease therapy, such as a cholinesterase inhibitor, a muscarinic agonist, an anti-oxidant, an anti-inflammatory, galantamine (Reminyl), tacrine (Cognex), selegiline, physostigmine, revistigmin, donepezil, (Aricept), rivastigmine (Exelon), metrifonate, milameline, xanomeline, saeluzole, acetyl-L-carnitine, idebenone, ENA-713, memric, quetiapine, neurestrol or neuromidal. Treating may comprise one or more of improvements in memory, cognition or learning, slowing the progression of symptoms or pathophysiology, improving quality of life, or increasing life span. The compound may be administered orally or by injection, including intravenously, intradermally, intraarterially, intraperitoneally, intracranially, intraarticularly, intraprostaticaly, intrapleurally, intramuscularly, or subcutaneously. The compound may be administered 1, 2, 3 or 4 times daily. The method may further comprise measuring cognition or memory in said subject prior to and/or after administration of said compound. The mammalian subject may be a human.

In yet another embodiment, there is provided a compound having a formula selected from:

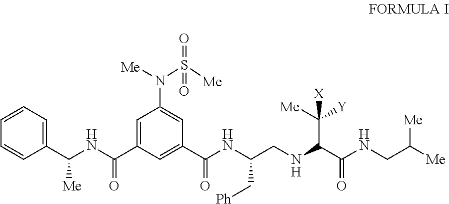

wherein X and Y are H or OH; or

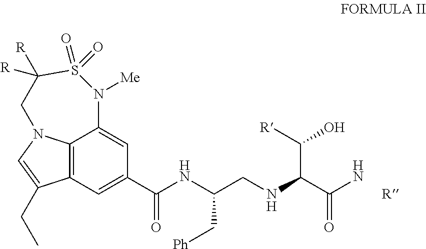

wherein each R, R′ and R′ are independently selected from C_{<8} alkyl, C_{<8} substituted alkyl, C_{<8} heterocycloalkyl, C_{<8} alkoxyalkyl, C_{<9} alkylamino, C_{<12} aryl, C_{<12} arylalkyl,
or a pharmaceutically acceptable salt or tautomer of any of the above formulas. The compound may have formula I, wherein X is H and Y is OH. Alternatively, the compound may have formula I, wherein X is H and Y is H. The compound may have formula II, wherein (i) R is H, R′ is —CH₃, and R″ is isobutyl, or (ii) wherein R is H, R′ is n-propyl, and R″ is isobutyl, or (iii) wherein R is H, R′ is isopropyl, and R″ is isobutyl, or (iv) wherein each R together form —CH₂—CH₂—, R′ is —CH₃, and R″ is isobutyl, or (v) wherein each R together form —CH₂—CH₂—, R′ is n-propyl, and R″ is isobutyl, or (vi) wherein each R together form —CH₂—CH₂—, R′ is isopropyl, and R″ is isobutyl, or (vii) wherein R′ is isopropyl.

Also provided is pharmaceutical composition comprising a compound as described above, formulated in a pharmaceutical buffer, diluent or excipient. The composition may be in a solid dosage form such as a tablet, a capsule or a powder. Alternatively, the composition is in a liquid dosage form.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Structures of β-secretase inhibitors 1-3.

FIG. 2. Structure of isophthalamide-derived β-secretase inhibitors 4-9.

FIG. 3. Structure of indole-derived β-secretase inhibitors 18-22.

FIG. 4. X-ray structure of 5 (green)-bound-β-secretase complex. Hydrogen bonds are shown in dotted lines (PDB ID: 4GID).

FIGS. 5A-C. Synthetic schemes.

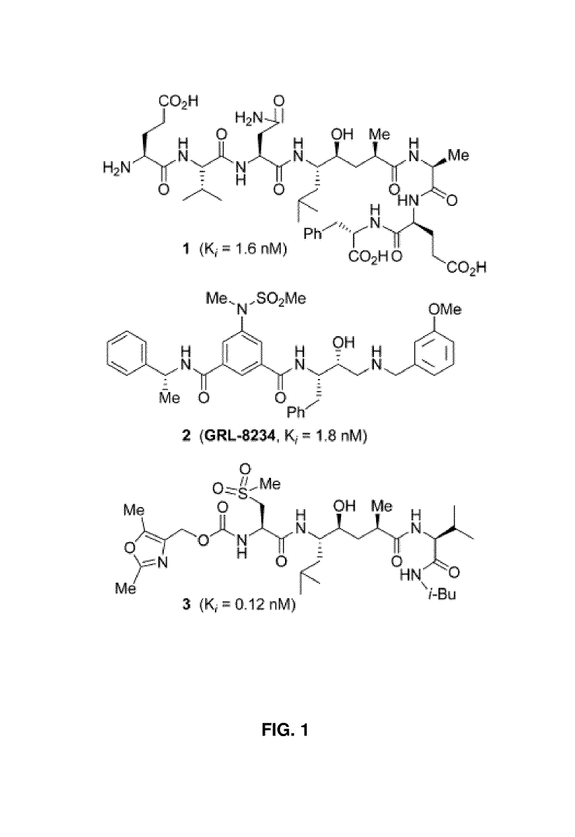

### DESCRIPTION OF ILLUSTRATIVE EMBODIMENTS

Aspartic proteases are a family of protease enzymes that use two aspartate residues for catalysis of the hydrolysis of their peptide substrates. In general, they have two highly-conserved aspartates in the active site and, usually but not always, their optimally active at an acidic pH. Aspartic proteases are involved in disease such as hypertension, HIV, tumorigenesis, peptic ulcer disease, amyloid disease, malaria and common fungal infections such as candidiasis.

Eukaryotic aspartic proteases include pepsins, cathepsins, and renins. They have a two-domain structure, arising from ancestral gene duplication and fusion. Each domain contributes a catalytic Asp residue, with an extended active site cleft localized between the two lobes of the molecule. One lobe has probably evolved from the other through a gene duplication event in the distant past. In modern-day enzymes, although the three-dimensional structures are very similar, the amino acid sequences are more divergent, except for the catalytic site motif, which is highly conserved. The presence and position of disulfide bridges are other somewhat conserved features of aspartic peptidases.

In an attempt to design small molecule inhibitors with improved selectivity and cellular activity exploiting this unique interaction, the have further explored β-secretase inhibitors with a reduced amide isostere and incorporated functionality to improve potency and selectivity. The basic amine functionality in the reduced amide isostere may also improve cell permeability (Labby et al., 2012). Herein, the inventors report structure-based design and synthesis of very potent and exceptionally selective inhibitors with excellent cellular inhibitory properties. A protein-ligand X-ray structure provided important molecular insight into the specific cooperative ligand-binding site interactions for selectivity. The identity of these compounds, and their use in treating Alzheimer's Disease, are discussed in detail below.

### I. ALZHEIMER'S DISEASE AND Aβ PEPTIDE

Alzheimer's disease (AD) is a degenerative disorder of the brain first described by Alios Alzheimer in 1907 after examining one of his patients who suffered drastic reduction in cognitive abilities and had generalized dementia. It is the leading cause of dementia in elderly persons. AD patients have increased problems with memory loss and intellectual functions which progress to the point where they cannot function as normal individuals. With the loss of intellectual skills the patients exhibit personality changes, socially inappropriate actions and schizophrenia. AD is devastating for both victims and their families given that there is no effective palliative or preventive treatment for the inevitable neurodegeneration.

AD is associated with neuritic plaques measuring up to 200 μm in diameter in the cortex, hippocampus, subiculum, hippocampal gyrus, and amygdala. One of the principal constituents of neuritic plaques is amyloid, which is stained by Congo Red (Kelly, 1984)). Amyloid plaques stained by Congo Red are extracellular, pink or rust-colored in bright field, and birefringent in polarized light. The plaques are composed of polypeptide fibrils and are often present around blood vessels, reducing blood supply to various neurons in the brain.

Various factors such as genetic predisposition, infectious agents, toxins, metals, and head trauma have all been suggested as possible mechanisms of AD neuropathy. Available evidence strongly indicates that there are distinct types of genetic predispositions for AD. First, molecular analysis has provided evidence for mutations in the amyloid precursor protein (APP) gene in certain AD-stricken families (Goate et al., 1991; Murrell et al., 1991; Chartier-Harlin et al., 1991 and Mullan et al., 1992). Additional genes for dominant forms of early onset AD reside on chromosome 14 and chromosome 1 (Rogaev et al., 1995; Levy-Lahad et al., 1995 and Sherrington et al., 1995). Another loci associated with AD resides on chromosome 19 and encodes a variant form of apolipoprotein E (Corder, 1993).

Amyloid plaques are abundantly present in AD patients and in Down's Syndrome individuals surviving to the age of 40. The overexpression of APP in Down's Syndrome is recognized as a possible cause of the development of AD in Down's patients over thirty years of age (Rumble et al., 1989 and Mann et al., 1989). The plaques are also present in the normal aging brain, although at a lower number. These plaques are made up primarily of the amyloid f3 peptide (Aβ; sometimes also referred to in the literature as β-amyloid peptide or f3 peptide) (Glenner and Wong, 1984), which is also the primary protein constituent in cerebrovascular amyloid deposits. The amyloid is a filamentous material that is arranged in β-pleated sheets. Aβ is a hydrophobic peptide comprising up to 43 amino acids.

The determination of its amino acid sequence led to the cloning of the APP cDNA (Kang et al., 1987; Goldgaber et al., 1987; Robakis et al., 1987 and Tanzi et al., 1988) and genomic APP DNA (Lemaire et al., 1989 and Yoshikai et al., 1990). A number of forms of APP cDNA have been identified, including the three most abundant forms, APP695, APP751, and APP770. These forms arise from a single precursor RNA by alternate splicing. The gene spans more than 175 kb with 18 exons (Yoshikai et al., 1990). APP contains an extracellular domain, a transmembrane region and a cytoplasmic domain. Aβ consists of up to 28 amino acids just outside the hydrophobic transmembrane domain and up to 15 residues of this transmembrane domain. Aβ is normally found in brain and other tissues such as heart, kidney and spleen. However, Aβ deposits are usually found in abundance only in the brain.

(Van Broeckhaven et al., 1990), have demonstrated that the APP gene is tightly linked to hereditary cerebral hemorrhage with amyloidosis (HCHWA-D) in two Dutch families. This was confirmed by the finding of a point mutation in the APP coding region in two Dutch patients (Levy et al., 1990). The mutation substituted a glutamine for glutamic acid at position 22 of the Aβ (position 618 of APP695, or position 693 of APP770). In addition, certain families are genetically predisposed to Alzheimer's disease, a condition referred to as familial Alzheimer's disease (FAD), through mutations resulting in an amino acid replacement at position 717 of the full length protein (Goate et al., 1991; Murrell et al., 1991 and Chartier-Harlin et al., 1991). These mutations co-segregate with the disease within the families and are absent in families with late-onset AD. This mutation at amino acid 717 increases the production of the Aβ₁₋₄₂ form of Aβ from APP (Suzuki et al., 1994). Another mutant form contains a change in amino acids at positions 670 and 671 of the full length protein (Mullan et al., 1992). This mutation to amino acids 670 and 671 increases the production of total Aβ from APP (Citron et al., 1992).

APP is processed in vivo at three sites. The evidence suggests that cleavage at the β-secretase site by a membrane associated metalloprotease is a physiological event. This site is located in APP 12 residues away from the lumenal surface of the plasma membrane. Cleavage of the β-secretase site (28 residues from the plasma membrane's lumenal surface) and the β-secretase site (in the transmembrane region) results in the 40/42-residue β-amyloid peptide (Aβ), whose elevated production and accumulation in the brain are the central events in the pathogenesis of Alzheimer's disease (for review, see Selkoe, 1999). Presenilin 1, another membrane protein found in human brain, controls the hydrolysis at the APP β-secretase site and has been postulated to be itself the responsible protease (Wolfe et al., 1999). Presenilin 1 is expressed as a single chain molecule and its processing by a protease, presenilinase, is required to prevent it from rapid degradation (Thinakaran et al., 1996 and Podlisny et al., 1997). The identity of presenilinase is unknown. The in vivo processing of the β-secretase site is thought to be the rate-limiting step in Aβ production (Sinha & Lieberburg, 1999), and is therefore a strong therapeutic target.

The design of inhibitors effective in decreasing amyloid plaque formation is dependent on the identification of the critical enzyme(s) in the cleavage of APP to yield the 42 amino acid peptide, the Aβ₁₋₄₂ form of Aβ. Although several enzymes have been identified, it has not been possible to produce active enzyme. Without active enzyme, one cannot confirm the substrate specificity, determine the subsite specificity, nor determine the kinetics or critical active site residues, all of which are essential for the design of inhibitors.

### II. MEMAPSIN 2

Memapsin 2 (membrane-associated aspartic protease 2), or β-secretase 1 (BACE1; also known as β-site APP cleaving enzyme 1; β-site amyloid precursor protein cleaving enzyme 1; aspartyl protease 2 (ASP2)) is an enzyme that in humans is encoded by the BACE1 gene. It is an aspartic-acid protease important in the formation of myelin sheaths in peripheral nerve cells. It is a transmembrane protein containing two active site aspartate residues in its extracellular protein domain and may function as a dimer.

Memapsin 2 has been shown to a key protease involved in the production in human brain of β-amyloid peptide from β-amyloid precursor protein (for review, see Selkoe, 1999). It is now generally accepted that the accumulation of β-amyloid peptide in human brain is a major cause for the Alzheimer's Disease. Inhibitors specifically designed for human memapsin 2 should inhibit or decrease the formation of β-amyloid peptide and the progression of the Alzheimer's Disease.

Memapsin 2 belongs to the aspartic protease family. It is homologous in amino acid sequence to other eukaryotic aspartic proteases and contains motifs specific to that family. These structural similarities predict that memapsin 2 and other eukaryotic aspartic proteases share common catalytic mechanism (Davies, 1990). The most successful inhibitors for aspartic proteases are mimics of the transition state of these enzymes. These inhibitors have substrate-like structure with the cleaved planar peptide bond between the carbonyl carbon and the amide nitrogen replaced by two tetrahedral atoms, such as hydroxyethylene [—CH(OH)—CH₂—], which was originally discovered in the structure of pepstatin (Marciniszyn et al., 1976).

However, for clinical use, it is preferable to have small molecule inhibitors which will pass through the blood brain barrier and which can be readily synthesized. It is also desirable that the inhibitors are relatively inexpensive to manufacture and that they can be administered orally. Screening of thousands of compounds for these properties would require an enormous effort. To rationally design memapsin 2 inhibitors for treating Alzheimer's disease, it will be important to know the three-dimensional structure of memapsin 2, especially the binding mode of an inhibitor in the active site of this protease.

### III. INHIBITORS OF MEMAPSIN 2

### A. Definitions

When used in the context of a chemical group, “hydrogen” means —H; “hydroxy” means —OH; “oxo” means ═O; “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “ [symbol] ” represents a single bond or a double bond. Thus, for example, the structure

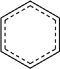

includes the structures

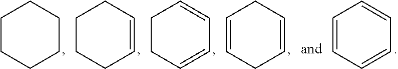

As will be understood by a person of skill in the art, no one such ring atom forms part of more than one double bond. The symbol “ [symbol] ”, when drawn perpendicularly across a bond indicates a point of attachment of the group. It is noted that the point of attachment is typically only identified in this manner for larger groups in order to assist the reader in rapidly and unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the conformation (e.g., either R or S) or the geometry is undefined (e.g., either E or Z).

Any undefined valency on an atom of a structure shown in this application implicitly represents a hydrogen atom bonded to the atom. When a group “R” is depicted as a “floating group” on a ring system, for example, in the formula:

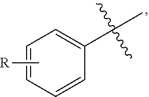

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

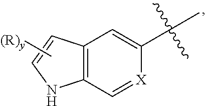

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the following parenthetical subscripts further define the group/class as follows: “(Cn)” defines the exact number (n) of carbon atoms in the group/class. “(C≦n)” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “saturated” as used herein means the compound or group so modified has no carbon-carbon double and no carbon-carbon triple bonds, except as noted below. The term does not preclude carbon-heteroatom multiple bonds, for example a carbon oxygen double bond or a carbon nitrogen double bond. Moreover, it does not preclude a carbon-carbon double bond that may occur as part of keto-enol tautomerism or imine/enamine tautomerism.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl). Where the term “aliphatic” is used without the “substituted” modifier, then only carbon and hydrogen atoms are present. When the term is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “alkyl” when used without the “substituted” modifier refers to a monovalent saturated aliphatic group with a carbon atom as the point of attachment, a linear or branched, cyclo, cyclic or acyclic structure, and no atoms other than carbon and hydrogen. Thus, as used herein cycloalkyl is a subset of alkyl. The groups —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and

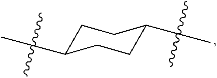 [SMILES: *C1CCC(*)CC1]

are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: ═CH₂, ═CH(CH₂CH₃), and ═C(CH₃)₂. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which one or more hydrogen atoms has been substituted with a halo group and no other atoms aside from carbon, hydrogen and halogen are present. The group, —CH₂Cl is a non-limiting examples of a haloalkyl. An “alkane” refers to the compound H—R, wherein R is alkyl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups. An “alkane” refers to the compound H—R, wherein R is alkyl.

The term “alkenyl” when used without the “substituted” modifier refers to an monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

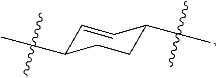 [SMILES: *C1C=CC(*)CC1]

are non-limiting examples of alkenediyl groups. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups, —CH═CHF, —CH═CHCl and —CH═CHBr, are non-limiting examples of substituted alkenyl groups. An “alkene” refers to the compound H—R, wherein R is alkenyl.

The term “alkynyl” when used without the “substituted” modifier refers to an monovalent unsaturated aliphatic group with a carbon atom as the point of attachment, a linear or branched, cyclo, cyclic or acyclic structure, at least one carbon-carbon triple bond, and no atoms other than carbon and hydrogen. As used herein, the term alkynyl does not preclude the presence of one or more non-aromatic carbon-carbon double bonds. The groups, —C≡CH, —C≡CCH₃, and —CH₂C≡CCH₃, are non-limiting examples of alkynyl groups. When alkynyl is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. An “alkyne” refers to the compound H—R, wherein R is alkynyl.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl group (carbon number limitation permitting) attached to the first aromatic ring or any additional aromatic ring present. Non-limiting examples of aryl groups include phenyl (Ph), methylphenyl, (dimethyl)phenyl, —C₆H₄—CH₂CH₃ (ethylphenyl), naphthyl, and the monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl group (carbon number limitation permitting) attached to the first aromatic ring or any additional aromatic ring present. If more than one ring is present, the rings may be fused or unfused. Non-limiting examples of arenediyl groups include:

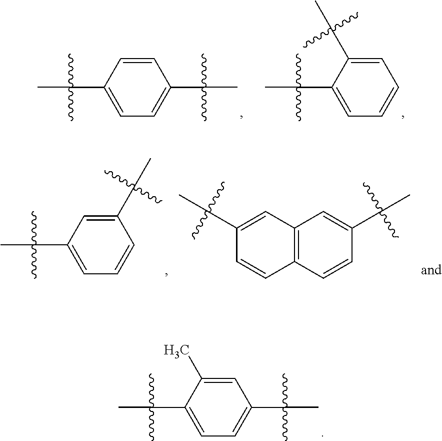

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. An “arene” refers to the compound H—R, wherein R is aryl.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn) and 2-phenyl-ethyl. When the term is used with the “substituted” modifier one or more hydrogen atom from the alkanediyl and/or the aryl has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. As used herein, the term does not preclude the presence of one or more alkyl, aryl, and/or aralkyl groups (carbon number limitation permitting) attached to the aromatic ring or aromatic ring system. If more than one ring is present, the rings may be fused or unfused. Non-limiting examples of heteroaryl groups include furanyl, imidazolyl, indolyl, indazolyl (Im), isoxazolyl, methylpyridinyl, oxazolyl, phenylpyridinyl, pyridinyl, pyrrolyl, pyrimidinyl, pyrazinyl, quinolyl, quinazolyl, quinoxalinyl, triazinyl, tetrazolyl, thiazolyl, thienyl, and triazolyl. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. As used herein, the term does not preclude the presence of one or more alkyl, aryl, and/or aralkyl groups (carbon number limitation permitting) attached to the aromatic ring or aromatic ring system. If more than one ring is present, the rings may be fused or unfused. Non-limiting examples of heteroarenediyl groups include:

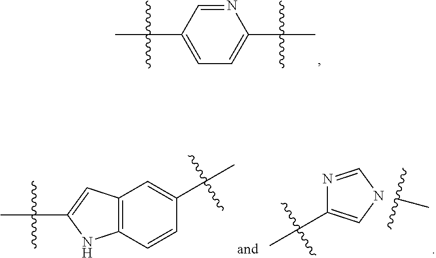

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. As used herein, the term does not preclude the presence of one or more alkyl groups (carbon number limitation permitting) attached to the ring or ring system. As used herein, the term does not preclude the presence of one or more double bonds in the ring or ring system, provided that the resulting groups remains non-aromatic. If more than one ring is present, the rings may be fused or unfused. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. When the term “heterocycloalkyl” used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “acyl” when used without the “substituted” modifier refers to the group —C(O)R, in which R is a hydrogen, alkyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄—CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. When either of these terms are used with the “substituted” modifier one or more hydrogen atom (including a hydrogen atom directly attached the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples of alkoxy groups include: —OCH₃ (methoxy), —OCH₂CH₃ (ethoxy), —OCH₂CH₂CH₃, —OCH(CH₃)₂ (isopropoxy), —OCH(CH₂)₂, —O-cyclopentyl, and —O-cyclohexyl. The terms “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The term “alcohol” corresponds to an alkane, as defined above, wherein at least one of the hydrogen atoms has been replaced with a hydroxy group.

The term “alkylamino” when used without the “substituted” modifier refers to the group —NHR, in which R is an alkyl, as that term is defined above. Non-limiting examples of alkylamino groups include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “alkoxyamino”, “alkenylamino”, “alkynylamino”, “arylamino”, “aralkylamino”, “heteroarylamino”, “heterocycloalkylamino” and “alkylsulfonylamino” when used without the “substituted” modifier, refers to groups, defined as —NHR, in which R is alkoxy, alkenyl, alkynyl, aryl, aralkyl, heteroaryl, heterocycloalkyl, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. The term “alkylaminodiyl” refers to the divalent group —NH-alkanediyl-, —NH-alkanediyl-NH—, or -alkanediyl-NH-alkanediyl-. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups —NHC(O)OCH₃ and —NHC(O)NHCH₃ are non-limiting examples of substituted amido groups.

The term “alkylphosphate” when used without the “substituted” modifier refers to the group —OP(O)(OH)(OR), in which R is an alkyl, as that term is defined above. Non-limiting examples of alkylphosphate groups include: —OP(O)(OH)(OMe) and —OP(O)(OH)(OEt). The term “dialkylphosphate” when used without the “substituted” modifier refers to the group —OP(O)(OR)(OR′), in which R and R′ can be the same or different alkyl groups, or R and R′ can be taken together to represent an alkanediyl. Non-limiting examples of dialkylphosphate groups include: —OP(O)(OMe)₂, —OP(O)(OEt)(OMe) and —OP(O)(OEt)₂. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The terms “alkylsulfonyl” and “alkylsulfinyl” when used without the “substituted” modifier refers to the groups —S(O)₂R and —S(O)R, respectively, in which R is an alkyl, as that term is defined above. The terms “alkenylsulfonyl”, “alkynylsulfonyl”, “aryl sulfonyl”, “aralkylsulfonyl”, “heteroarylsulfonyl”, and “heterocycloalkylsulfonyl” are defined in an analogous manner. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

As used herein, a “chiral auxiliary” refers to a removable chiral group that is capable of influencing the stereoselectivity of a reaction. Persons of skill in the art are familiar with such compounds, and many are commercially available.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methane-sulfonates, ethanesulfonates, benzenesulfonates, p-toluenesulfonates, cyclohexyl-sulfamates, quinates, esters of amino acids, and the like. Similarly, a compound comprising an amine group may be administered as an amide that is converted by hydrolysis in vivo to the amine compound.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2n, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diasteromers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≦15%, more preferably ≦10%, even more preferably ≦5%, or most preferably ≦1% of another stereoisomer(s).

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

The compounds provided by the present disclosure are shown, for example, above in the summary of the invention section and in the claims below. They may be made using the methods outlined in the Examples section. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Compounds of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration.

Compounds of the invention may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

Compounds of the present invention may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### B. Compounds of the Present Invention

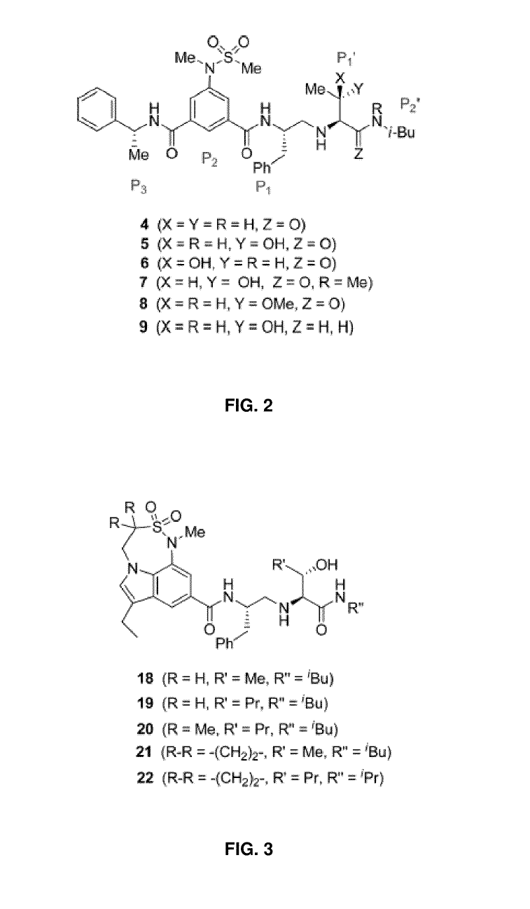

Compounds of the present invention are shown above in the Summary of the Invention, in the claims, FIG. 2, FIG. 3 and in Table 1.

Compounds of the invention may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

Compounds of the present invention may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### C. Synthetic Schemes

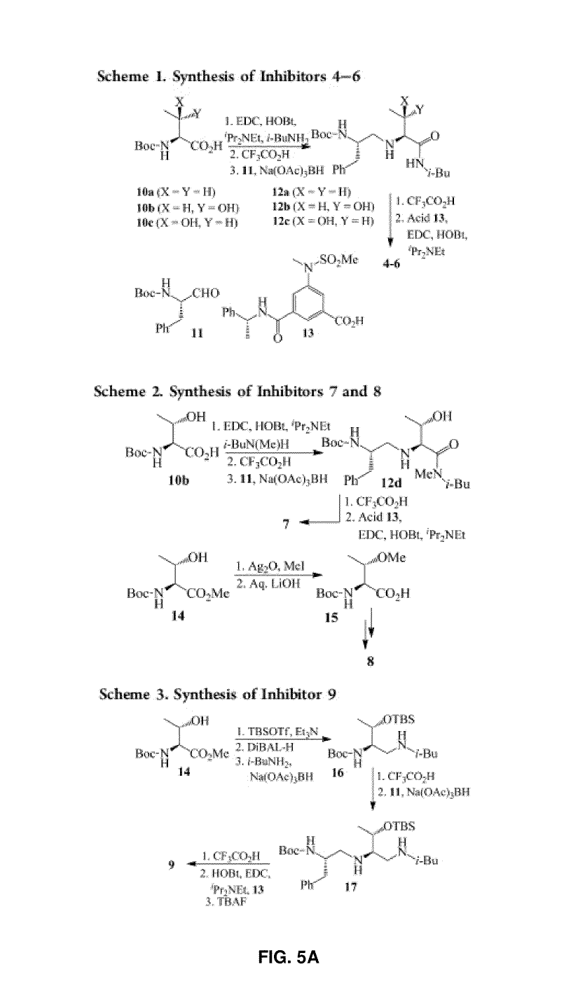

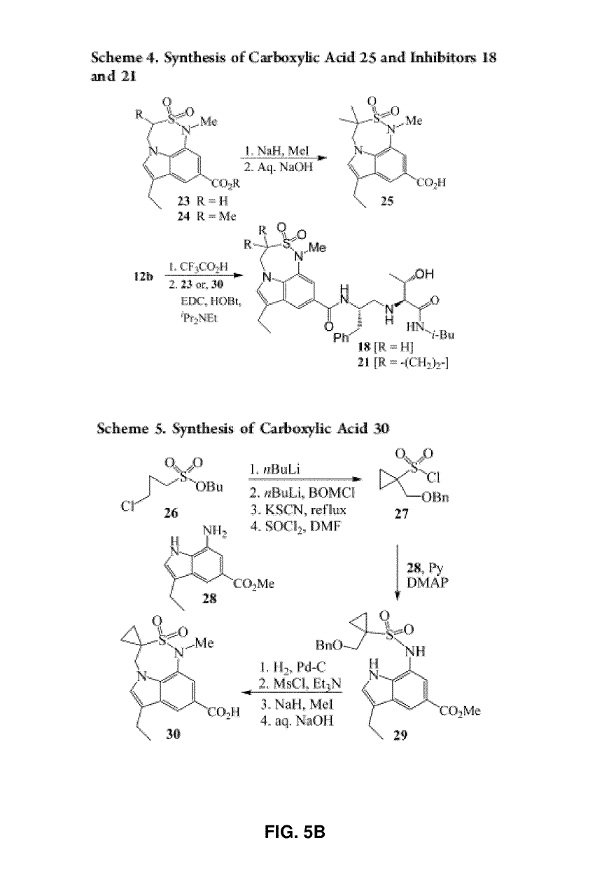

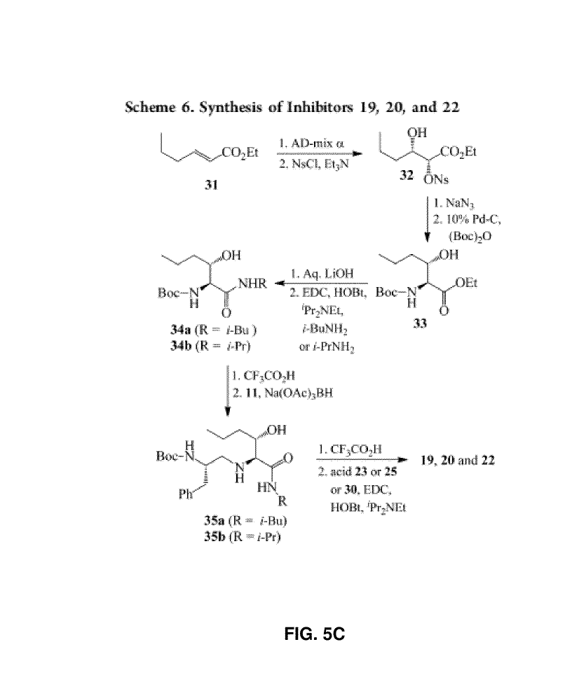

The synthesis of inhibitors 4-6 is shown in Scheme 1 (FIG. 5A). Amino acids 10a-c were coupled with isobutyl amine in the presence of EDC, HOBt, and iPr2NEt to afford the corresponding amides in 71-91% yield. Removal of the Boc group with trifluoroacetic acid followed by reductive amination of the resulting amine with aldehyde 11¹⁷ provided compounds 12a-c in 49-76% yields. Treatment of 12a-c with trifluoroacetic acid followed by coupling with acid 13 107 18 provided the inhibitors 4-6 in 68-75% yields.

The synthesis of inhibitors 7-9 is shown in Schemes 2 and 3 (FIG. 5A). Coupling of Boc-protected allothreonine derivative 10b with N-isobutyl-N-methyl amine using EDC, HOBt, and iPr2NEt followed by Boc removal using trifluoroacetic acid afforded (2S,3S)-2-amino-3-hydroxy-N-isobutyl-N-methyl-butanamide in 49% yield over two steps. Reductive amination of this amine with known aldehyde 11 produced 12d in 30% yield. Inhibitor 7 was synthesized in 61% yield from 12d by Boc removal using trifluoroacetic acid followed by coupling of the resulting amine with the known acid 13 (Scheme 2). As shown in Scheme 2, inhibitor 8 was prepared from 15 following the similar reaction sequence utilized for the synthesis of inhibitors 4-7 (Schemes 1 and 2). Compound 15, in turn, has been synthesized by O-122 methylation of 14 using MeI and Ag2O19 followed by hydrolysis using aqueous LiOH.

The synthesis of inhibitor 9 has been carried out as shown in Scheme 3. Treatment of 14 with TBSOTf in the presence of Et3N followed by reaction with DiBALH provided the corresponding aldehyde. Reductive amination of this crude aldehyde with isobutyl amine afforded 16 in 66% yield over three steps. Removal of the Boc group using trifluoroacetic acid followed by reductive amination of the resulting amine with the aldehyde 11 afforded compound 17.

Reaction of compound 17 with trifluoroacetic acid followed by coupling with acid 13 using EDC, HOBt, and iPr2NEt provided TBS-protected inhibitor, which upon treatment with TBAF gave the inhibitor 9 in 46% yield over three steps. On the basis of the results obtained from these inhibitors, the inventors have directed their attention to reduce the peptidic nature of 5 by keeping all of the key hydrogen-bonding interactions of the prime region intact. In this direction, the inventors have designed and synthesized the inhibitors 18-22 (FIG. 3) containing 7,6,5-tricyclic indole moieties as P2 ligands.

For the synthesis of inhibitor 18, known indolecarboxylic acid 23 was prepared as described in the literature.20 The synthesis of α,α-dimethyl tricyclic indole derivative 25, present in inhibitor 20, was synthesized as shown in Scheme 4 (FIG. 5B). Reaction of the known tricyclic indole derivative 24 (Hubert et al., 2004) with MeI in the presence of NaH provided the corresponding α,α-dimethyl derivative. Saponification of the resulting ester with aqueous NaOH afforded α,α-dimethylindole carboxylic acid 25 (37% yield over two steps). Inhibitor 18 was synthesized by treatment of Boc derivative 12b with trifluoroacetic acid followed by coupling of the resulting amine with acid 23 (Charrier et al., 2008) (68% yield). Similarly, coupling of the above amine with cyclopropyl indole derivative 30 produced inhibitor 21.

The synthesis of tricyclic indole derivative in inhibitors 21 and 22 has been carried out from butyl 3-chloropropanesul-fonate 26 as shown in Scheme 5 (FIG. 5B). In a one-pot two-step reaction, 26 was treated first with BuLi followed by treatment again with BuLi, and BOMCl provided the corresponding butyl 1-(benzyloxymethyl)cyclopropanesulfonate in 80% yield. Hydrolysis of this sulfonate with KSCN followed by refluxing of the resulting potassium sulfonate with SOCl2 afforded sulfonyl chloride 27 in a 91% yield over two steps. Reaction of indole derivative 28 (Charrier et al., 2006) with 27 in the presence of pyridine and a catalytic amount of DMAP furnished sulfonamide 29 in 74% yield. Hydrogenolysis of 29, followed by mesylation of the resulting alcohol using mesyl chloride and Et3N, afforded the corresponding mesylate. This mesylate was subjected to a one-pot cyclization and N-methylation using NaH and MeI followed by hydrolysis in the presence of aqueous NaOH to obtain the desired carboxylic acid 30 in 65% yield over two steps.

The synthesis of (2S,3S)-2-amino-3-hydroxy-N-isobutyl (or isopropyl)hexanamide moiety present in the prime region of the inhibitors 19, 20, and 22 has been carried out as shown in Scheme 6 (FIG. 5C). Asymmetric dihydroxylation of 31 using AD-mix a followed by reaction of the resulting dihydroxy compound with p-nitrobenzenesulfonyl chloride in the presence of Et3N afforded nosylate 32 in 32% yield over two steps (Fleming and Sharpless, 1991). Treatment of 32 with NaN3 followed by hydrogenation of the corresponding azide using 10% Pd—C in the presence of (Boc)2O afforded 33 in 80% yield. Hydrolysis using aqueous LiOH followed by coupling with isobutyl amine and isopropyl amine using EDC, HOBt, and iPr2NEt afforded 34a and 34b, respectively. Boc removal using trifluoroacetic acid followed by reductive amination of the resulting amine with aldehyde 11 gave 35a and 35b in 49 and 74% yields, respectively. Inhibitors 19, 20, and 22 were synthesized by coupling of the amine, obtained by Boc removal of 35a and 35b using trifluoroacetic acid, with tricyclic indole derivatives 23 or 25 or 30.

### IV. TREATMENT OF ALZHEIMER'S DISEASE

### A. Formulations and Routes of Administration

In accordance with the present invention, patients with Alzheimer's Disease are treated with the compounds described herein. It will be necessary to prepare pharmaceutical compositions in a form appropriate for administration to a subject. The compositions will generally be prepared essentially free of pyrogens, as well as other impurities that could be harmful to humans or animals. One will generally desire to employ appropriate salts and buffers to render stable cells suitable for introduction into a patient. Aqueous compositions of the present invention comprise an effective amount of stable cells dispersed in a pharmaceutically acceptable carrier or aqueous medium, and preferably encapsulated.

The phrase “pharmaceutically or pharmacologically acceptable” refer to compositions that do not produce adverse, allergic, or other untoward reactions when administered to an animal or a human. As used herein, “pharmaceutically acceptable carrier” includes any and all solvents, dispersion media, antibacterial and antifungal agents, isotonic and absorption delaying agents and the like. As used herein, this term is particularly intended to include biocompatible implantable devices and encapsulated cell populations. The use of such media and agents for pharmaceutically active substances is well know in the art. Except insofar as any conventional media or agent is incompatible with the compositions of the present invention, its use in therapeutic compositions is contemplated. Supplementary active ingredients also can be incorporated into the compositions.

Under ordinary conditions of storage and use, the cell preparations may further contain a preservative to prevent growth of microorganisms. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

The compositions will advantageously be administered orally or by injection, including intravenously, intradermally, intraarterially, intraperitoneally, intracranially, intraarticularly, intraprostaticaly, intrapleurally, intramuscularly, subcutaneously, or by other method or any combination of the forgoing as would be known to one of ordinary skill in the art.

As will be recognized by those in the field, a “therapeutically effective amount” refers to an mount of such that, when provided to a subject in accordance with the disclosed and claimed methods effectsone of the following biological activities: treatment of any aspect or symptom Alzheimer's Disease, including improvements in memory, cognition or learning, slowing the progression of symptoms or pathophysiology, improving quality of life, or increasing life span, in a subject diagnosed with or otherwise having Alzheimer's Disease.

As understood in the art, such therapeutically effective amount will vary with many factors including the age and weight of the patient, the patient's physical condition, the condition to be treated, and other factors. An effective amount of the disclosed compounds will also vary with the particular combination administered. However, typical doses may contain from a lower limit of about 1 μg, 5 μg, 10 μg, 50 μg to 100 μg to an upper limit of about 100 μg, 500 μg, 1 mg, 5 mg, 10 mg, 50 mg or 100 mg of the pharmaceutical compound per day. Also contemplated are other dose ranges such as 0.1 μg to 1 mg of the compound per dose. The doses per day may be delivered in discrete unit doses, provided continuously in a 24 hour period or any portion of that the 24 hours. The number of doses per day may be from 1 to about 4 per day, although it could be more. Continuous delivery can be in the form of continuous infusions. The terms “QID,” “TID,” “BID” and “QD” refer to administration 4, 3, 2 and 1 times per day, respectively. Exemplary doses and infusion rates include from 0.005 nmol/kg to about 20 nmol/kg per discrete dose or from about 0.01/pmol/kg/min to about 10 pmol/kg/min in a continuous infusion. These doses and infusions can be delivered by intravenous administration (i.v.) or subcutaneous administration (s.c.). Exemplary total dose/delivery of the pharmaceutical composition given i.v. may be about 2 μg to about 8 mg per day, whereas total dose/delivery of the pharmaceutical composition given s.c. may be about 6 μg to about 6 mg per day.

The disclosed compounds may be administered, for example, at a daily dosage of, for example: from about 0.01 mg/kg to about 100 mg/kg; from about 0.01 mg/kg to about 80 mg/kg; from about 0.01 mg/kg to about 70 mg/kg; from about 0.01 mg/kg to about 60 mg/kg; from about 0.01 mg/kg to about 50 mg/kg; from about 0.01 mg/kg to about 40 mg/kg; from about 0.01 mg/kg to about 30 mg/kg; from about 0.01 mg/kg to about 25 mg/kg; from about 0.01 mg/kg to about 20 mg/kg; from about 0.01 mg/kg to about 15 mg/kg; from about 0.01 mg/kg to about 10 mg/kg; from about 0.01 mg/kg to about 5 mg/kg; from about 0.01 mg/kg to about 3 mg/kg; from about 0.01 mg/kg to about 1 mg/kg; from about 0.01 mg/kg to about 0.3 mg/kg from about 100 mg/kg to about 90 mg/kg; from about 100 mg/kg to about 80 mg/kg; from about 100 mg/kg to about 70 mg/kg; from about 100 mg/kg to about 60 mg/kg; from about 100 mg/kg to about 50 mg/kg; from about 100 mg/kg to about 40 mg/kg; from about 85 mg/kg to about 10 mg/kg; from about 75 mg/kg to about 20 mg/kg; from about 65 mg/kg to about 30 mg/kg; from about 55 mg/kg to about 35 mg/kg; or from about 55 mg/kg to about 45 mg/kg. Administration may be by injection of a single dose or in divided doses.

The term “unit dose” refers to a physically discrete unit suitable for use in a subject, each unit containing a predetermined quantity of the composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

### B. Combination Therapy

In another embodiment, the inhibitors of the present invention may be used in combination with other agents to improve or enhance the therapeutic effect of either. This process may involve administering both agents to the patient at the same time, either as a single composition or pharmacological formulation that includes both agents, or by administering two distinct compositions or formulations, wherein one composition includes an inhibitor of the present invention and the other includes the second agent(s).

The therapy of the present invention also may precede or follow the second agent treatment by intervals ranging from minutes to weeks. In embodiments where the other agent and the inhibitor of the present invention are administered separately, one may prefer that a significant period of time did not expire between the time of each delivery, such that the agent and present inhibitor would still be able to exert an advantageously combined effect. In such instances, it is contemplated that one may administer both modalities within about 12-24 hours of each other and, more preferably, within about 6-12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In other embodiments, it may be desirable to alternate the compositions so that the subject is not tolerized.

Various additional combinations may be employed, inhibitor of the present invention is “A” and the secondary agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is expected that the treatment cycles would be repeated as necessary.

Various drugs for the treatment of AD are currently available as well as under study and regulatory consideration. The drugs generally fit into the broad categories of cholinesterase inhibitors, muscarinic agonists, anti-oxidants or anti-inflammatories. Galantamine (Reminyl), tacrine (Cognex), selegiline, physostigmine, revistigmin, donepezil, (Aricept), rivastigmine (Exelon), metrifonate, milameline, xanomeline, saeluzole, acetyl-L-carnitine, idebenone, ENA-713, memric, quetiapine, neurestrol and neuromidal are just some of the drugs proposed as therapeutic agents for AD.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

General.

All anhydrous solvents were obtained according to the following procedures: diethyl ether and tetrahydrofuran (THF) were distilled from sodium/benzophenone under argon; dichloromethane was from calcium hydride. Other solvents were used without purification. All moisture-sensitive reactions were carried out in flame-dried flasks under an argon atmosphere. Reactions were monitored by thin-layer chromatography (TLC) using Silicycle 60A-F254 silica gel precoated plates. Flash column chromatography was performed using Silicycle 230-400 mesh silica gel. Yields refer to chromatographically and spectroscopically pure compounds. 1H NMR and 13C NMR spectra were recorded on a Varian Inova-300 (300 and 75 MHz, respectively), Bruker Avance ARX-400 (400 and 100 MHz), and Bruker Avance DRX-500 (500 and 125 MHz). High- and low-resolution mass spectra were carried out by the Mass Spectroscopy Center at Purdue University. The purity of all test compounds was determined by HRMS and HPLC analysis. All test compounds showed ≧95% purity.

### Synthesis of Compound 12a

To a mixture of (S)-2-[(tert-butoxycarbonyl)amino]butanoic acid 10a (2.3 mmol, 0.47 g) and iPr2NEt (2.76 mmol, 0.48 mL) in CH₂Cl₂ (12 mL), HOBt.H₂O (2.76 mmol, 0.37 g), isobutyl amine (2.76 mmol, 0.27 mL), and EDC.HCl (2.76 mmol, 0.53 g) were added simultaneously at 23° C., and the resulting mixture was stirred for 17 hr at 23° C. The reaction mixture was quenched with saturated aqueous NaHCO₃ solution and extracted with CH₂Cl₂. The combined extracts were dried over anhydrous Na₂SO₄, filtered, and concentrated under reduced pressure. The resulting residue was purified by silica gel column chromatography (1-3% MeOH/CH₂Cl₂) to furnish (S)-tert-butyl [1-(isobutylamino)-oxobutan-2-yl]carbamate in 86% yield (0.51 g). ¹H NMR (400 348 MHz, CDCl₃): δ 6.24 (brs, ¹H), 5.08 (br, ¹H), 4.07-3.89 (m, ¹H), 3.18-2.96 (m, ²H), 1.92-1.70 (m, ²H), 1.62 (hept, J=7.3 Hz, ¹H), 1.43 (s, ⁹H), 0.93 (t, J=7.4 Hz, ³H), 0.89 (d, J=6.8 Hz, 6H).

To a solution of (S)-tert-butyl [1-(isobutylamino)-1-oxobutan-2-yl]carbamate (1.97 mmol, 0.51 g) in CH₂Cl₂ (9 mL), trifluoroacetic acid (3 mL) was added at 0° C., and the resulting mixture was stirred for 1.5 hr at 23° C. Excess trifluoroacetic acid and CH₂Cl₂ were removed under reduced pressure, and the resulting residue was purified by silica gel column chromatography [2-5% (5% NH₃/MeOH)/CH₂Cl₂] to furnish (S)-2-amino-N-isobutylbutanamide in 96% yield (0.298 g).

To a solution of the above (S)-2-amino-N-isobutylbutanamide (1.87 mmol, 0.296 g) and (S)-tert-butyl (1-oxo-3-phenylpropan-2-yl)-carbamate 11 [prepared from the corresponding Weinreb amide (2 mmol) following a similar literature procedure]7 in CH₂Cl₂ (20 mL), Na(OAc)₃BH (2.62 mmol, 0.55 g) was added at 0° C. The resulting mixture was stirred for 1 hr at 0° C. and 15 hr at 23° C. The reaction mixture was quenched with saturated aqueous NaHCO₃ solution and extracted with CH₂Cl₂. The combined extracts were dried over anhydrous Na₂SO₄, filtered, and concentrated under reduced pressure. The residue was purified by silica gel column chromatography (1-3% MeOH/CH₂Cl₂) to furnish the corresponding adduct 12a in 79% yield (0.58 g). ¹H NMR (400 MHz, CDCl₃): δ 7.34-7.26 (m, ²H), 7.25-7.11 (m, ⁴H), 4.50 (br, ¹H), 3.95 (brs, 1H), 3.10-2.96 (m, ²H), 2.92 (dd, J=7.3, 5.0 Hz, ¹H), 2.86-2.69 (m, ²H), 2.62 (dd, J=11.9, 4.3 Hz, ¹H), 2.52 (dd, J=12.0, 7.9 Hz, ¹H), 1.80-1.63 (m, ²H), 1.63-1.50 (m, ¹H), 1.41 (s, ⁹H), 0.92 (t, J=7.5 Hz, ³H), 0.85 (d, J=6.5 Hz, ⁶H).

### Synthesis of Inhibitor 4

To a solution of 12a (0.72 mmol, 0.282 g) in CH₂Cl₂ (9 mL), trifluoroacetic acid (3 mL) was added at 0° C. After the reaction mixture was stirred for 1 hr at 23° C., CH₂Cl₂ and trifluoroacetic acid were removed under reduced pressure. The resulting residue was purified by silica gel column chromatography [2-6% (5% NH₃/MeOH)/CH₂Cl₂] to furnish the corresponding amine in 93% yield (0.195 g).

To a solution of the above amine (0.125 mmol, 36.4 mg) in CH₂Cl₂ (10 mL), iPr2NEt (0.03 mL), HOBt.H₂O (0.16 mmol, 21.6 mg), (R)-3-(N-methylmethylsulfonamido)-5-[(1-phenylethyl)-carbamoyl]-benzoic acid 13 (0.16 mmol, 60.2 mg), and EDC.HCl (0.16 mmol, 30.7 mg) were added simultaneously at 23° C., and the resulting mixture was stirred for 14 hr at the same temperature. The reaction mixture was quenched with saturated aqueous NaHCO₃ solution and extracted with CH₂Cl₂. The combined extracts were dried over anhydrous Na₂SO₄, filtered, and concentrated under reduced pressure. The resulting residue was purified by silica gel column chromatography (1-3% MeOH/CH₂Cl₂) to furnish the inhibitor 4 in 73% yield (59.6 mg). ¹H NMR (400 MHz, CDCl₃): δ 8.26 (s, ¹H), 8.03 (s, ¹H), 7.98 (s, ¹H), 7.48 (d, J=7.8 Hz, ¹H), 7.39-7.34 (m, ²H), 7.33-7.24 (m, ⁵H), 7.24-7.18 (m, ³H), 6.71 (t, J=6.2 Hz, ¹H), 5.31 (p, J=7.5 Hz, ¹H), 4.40-4.26 (m, ¹H), 3.29 (s, ³H), 3.04-2.91 (m, ³H), 2.88-2.83 (m, ¹H), 2.81 (s, ³H), 2.81-2.73 (m, ²H), 2.55 (dd, J=12.3, 4.1 Hz, ¹H), 1.75-1.58 (m, ²H), 1.57 (d, J=7.0 Hz, ³H), 1.55-1.48 (m, ¹H), 0.89 (t, J=7.4 Hz, ³H), 0.77 (d, J=6.7 Hz, 3H), 0.76 (d, J=6.6 Hz, 3H). ¹³C NMR (100 MHz, CDCl₃): δ 174.15, 165.23, 164.53, 143.06, 142.23, 137.64, 135.65, 129.1, 128.62, 128.58, 127.93, 127.87, 127.33, 126.64, 126.23, 123.39, 65.20, 51.68, 50.24, 49.56, 46.41, 38.55, 37.84, 35.52, 28.36, 26.96, 21.60, 19.98, 19.95, 10.33. HRMS-ESI (m/z): [M+H]+ calcd for C₃₅H₄₈N₅O₅S, 650.3376; found, 650.3370.

### Synthesis of Compound 12b

tert-Butyl [(2S,3S)-3-hydroxy-1-(isobutylamino)-1-oxobutan-2-yl]carbamate was synthesized in 71% yield by coupling of (2S,3S)-2-[(tert-butoxycarbonyl)amino]-3-hydroxy-ybutanoic acid 10b with isobutyl amine in the presence of EDC, HOBt, and iPr2NEt as described for (S)-tert-butyl [1-(isobutylamino)-1-oxobutan-2-yl]carbamate. 1H NMR (400 MHz, CDCl₃): δ 6.47 (brs, ¹H), 5.53 (brs, ¹H), 4.10-3.75 (m, ³H), 3.23-3.09 (m, ¹H), 3.06-2.92 (m, ¹H), 1.78 (hept, J=6.7 Hz, ¹H), 1.45 (s, ⁹H), 1.29 (d, J=6.1 Hz, ³H), 0.91 (d, J=6.5 Hz, ⁶H).

Compound 12b was synthesized in 52% yield (for two steps) from tert-butyl [(2S,3S)-3-hydroxy-1-(isobutylamino)-1-oxobutan-2-yl]-carbamate following the procedure described for the synthesis of compound 12a. 1H NMR (400 MHz, CDCl₃): δ 7.35-7.27 (m, ³H), 7.26-7.19 (m, ¹H), 7.17 (d, J=7.4 Hz, ²H), 4.53 (d, J=8.9 Hz, ¹H), 4.05-3.86 (m, ²H), 3.39 (brs, ¹H), 3.13-3.00 (m, ²H), 2.99 (d, J=5.7 Hz, ¹H), 2.87-2.71 (m, ²H), 2.67 (dd, J=12.1, 4.4 Hz, ¹H), 2.58 (dd, J=12.1, 7.6 Hz, ¹H), 1.73 (hept, J=6.7 Hz, ¹H), 1.41 (s, ⁹H), 1.18 (d, J=6.5 Hz, ³H), 0.87 (d, J=7.1 Hz, ⁶H).

### Synthesis of Inhibitor 5

Inhibitor 5 has been prepared (yield 71%, over two steps) from 12b by boc deprotection followed by coupling with (R)-3-(N-methylmethylsulfonamido)-5-[(1-phenylethyl)-carbamoyl]benzoic acid 13 following a similar reaction procedure described for the inhibitor 4. ¹H NMR (400 MHz, CDCl³): δ 8.13 (s, ¹H), 7.93 (s, ¹H), 7.88 (s, ¹H), 7.39-7.27 (m, ⁶H), 7.26-7.14 (m, ⁶H), 5.27 (p, J=7.3 Hz, ¹H), 4.48-4.30 (m, ¹H), 4.04-3.94 (m, ¹H), 3.24 (s, ³H), 3.09 (d, J=4.3 Hz, ¹H), 3.02-2.88 (m, ³H), 2.88-2.77 (m, ¹H), 2.80 (s, ³H), 2.73 (dd, J=12.4, 3.7 Hz, ¹H) 2.64 (dd, J=12.3, 7.7 Hz, ¹H), 1.64 (hept, J=6.7 Hz 1H), 1.56 (d, J=6.9 Hz, 3H), 1.04 (d, J=6.2 Hz, ³H), 0.80 (d, J=6.8 Hz, ³H), 0.80 (d, J=6.8 Hz, ³H). ¹³C NMR (100 MHz, CDCl₃): δ 172.37, 166.01, 164.75, 142.91, 142.03, 137.54, 135.90, 135.77, 129.16, 128.65, 128.59, 127.89, 127.62, 127.43, 126.65, 126.23, 123.87, 68.21, 68.06, 52.16, 51.60, 49.66, 46.40, 39.00, 37.83, 35.56, 28.34, 21.65, 20.06, 18.21. HRMS-ESI (m/z): [M+H]+ calcd for C₃₅H₄₈N₅O₆S, 666.3325; found, 666.3321.

### Synthesis of Compound 12c

tert-Butyl [(2S,3R)-3-hydroxy-1-(isobutylamino)-1-oxobutan-2-yl]carbamate has been synthesized (yield 91%) by coupling of (2S,3R)-2-[(tert-butoxycarbonyl)amino]-3-hydroxybutanoic acid 10c with isobutyl amine in the presence of iPr2NEt, HOBt, and EDC as described for (S)-tert-butyl [1-(isobutylamino)-1-oxobutan-2-yl]carbamate. ¹H NMR (400 MHz, CDCl₃): δ 6.71 (brs, ¹H), 5.52 (d, J=7.6 Hz, ¹H), 4.45-4.30 (m, ¹H), 3.97 (dd, J=8.2, 1.9 Hz, ¹H), 3.22-3.08 (m, ¹H), 3.07-2.92 (m, ¹H), 1.77 (heptet, J=6.7 Hz, ¹H), 1.45 (s, ⁹H), 1.18 (d, J=6.4 Hz, ³H), 0.90 (d, J=6.9 Hz, ⁶H).

Compound 12c was prepared from tert-butyl [(2S,3R)-3-hydroxy-1-(isobutylamino)-1-oxobutan-2-yl]carbamate following the procedure described for the synthesis of compound 12a (yield 49% over two steps). ¹H NMR (400 MHz, CDCl₃): δ 7.37-7.26 (m, ²H), 7.25-7.13 (m, ⁴H), 4.59 (d, J=8.5 Hz, ¹H), 4.04-3.75 (m, ²H), 3.15-2.96 (m, ²H), 2.94 (d, J=5.2 Hz, ¹H), 2.80 (d, J=6.2 Hz, ²H), 2.70-2.55 (m, 457 ²H), 1.92 (br, ¹H), 1.74 (hept, J=6.7 Hz, ¹H), 1.40 (s, ⁹H), 1.18 (d, J=6.3 Hz, ³H), 0.87 (d, J=6.4 Hz, ⁶H).

### Synthesis of Inhibitor 6

Inhibitor 6 has been prepared (yield 75%, over two steps) from 12c by boc deprotection followed by coupling with (R)-3-(N-methylmethylsulfonamido)-5-[(1-phenylethyl)-carbamoyl]benzoic acid 13 following a similar reaction procedure described for the inhibitor 4. ¹H NMR (400 MHz, CDCl₃): δ 8.15 (s, ¹H), 7.92 (s, ¹H), 7.87 (s, ¹H), 7.48 (d, J=7.7 Hz, ¹H), 7.38-7.27 (m, ⁵H), 7.26-7.15 (m, ⁵H), 7.08 (t, J=5.9 Hz, ¹H), 5.27 (p, J=7.1 Hz, ¹H), 4.42-4.27 (m, ¹H), 3.79 (p, J=6.3 Hz, ¹H), 3.22 (s, ³H), 3.01-2.87 (m, ⁴H), 2.84 (dd, J=13.7, 7.7 Hz, ¹H), 2.78 (s, ³H), 2.71-2.59 (m, ²H), 1.69-1.59 (m, ¹H), 1.56 (d, J=7.0 Hz, ³H), 1.14 (d, J=6.2 Hz, ³H), 0.79 (d, J=6.4 Hz, ³H), 0.78 (d, J=6.6 Hz, ³H). ¹³C NMR (100 MHz, CDCl₃): δ 172.78, 165.82, 164.81, 143.03, 142.05, 137.58, 135.88, 135.77, 129.14, 128.61, 127.78, 127.64, 127.39, 126.64, 126.23, 123.80, 69.13, 68.38, 52.04, 50.90, 49.68, 46.54, 38.59, 37.79, 35.56, 28.33, 21.68, 20.03, 19.68. HRMS-ESI (m/z): [M+H]+ calc'd for C₃₅H₄₈N₅O₆S, 666.3325; found, 666.3335.

### Synthesis of Compound 12d

To a mixture of (2S,3S)-2-[(tert-butoxycarbonyl)amino]-3-hydroxybutanoic acid (10b) (1 mmol, 0.22 g) and iPr2NEt (1.2 mmol, 0.21 mL) in CH₂Cl₂ (5 mL) were added HOBt.H₂O (1.2 mmol, 0.162 g), N-isobutyl-N-methylamine (1.2 mmol, 0.14 mL), and EDC.HCl (1.2 mmol, 0.23 g) at 23° C., and the resulting reaction mixture was stirred for 15 hr at 23° C. The reaction mixture was quenched with saturated aqueous NaHCO₃ solution and extracted with CH₂Cl₂. The combined extracts were dried over anhydrous Na₂SO₄, filtered, and concentrated under reduced pressure. The residue was purified by silica gel column chromatography (1-3% MeOH/CH₂Cl₂) to obtain tert-butyl {(2S,3S)-3-hydroxy-1-[isobutyl-(methyl)amino]-1-oxobutan-2-yl}carbamate.

To a solution of the above tert-butyl {(2S,3S)-3-hydroxy-1-[isobutyl(methyl)amino]-1-oxobutan-2-yl}carbamate in CH₂Cl₂ (6 mL) was added trifluoroacetic acid (2 mL) at 0° C. The resulting reaction mixture was warmed to 23° C. and stirred for 1.5 hr at 23° C. The reaction mixture was concentrated under reduced pressure, and the resulting residue was purified by silica gel column chromatography [2-5% (5% NH₃/MeOH)/CH₂Cl₂] to furnish (2S,3S)-2-amino-3-hydroxy-N-isobutyl-N-methyl-butanamide in 49% (93.2 mg) yield over two steps. ¹H NMR (400 MHz, CDCl₃): δ 3.89-3.76 (m, ¹H), 3.67 and 3.64 (two doublets, J=4.7 Hz, 4.6 Hz, ¹H), 3.36 (dd, J=13.2, 8.0 Hz, ^{0.5}H), 3.19 (dd, J=14.4, 8.3 Hz, ^{0.5}H), 3.10 (dd, J=14.3, 7.0 Hz, ^{0.5}H), 3.05 (s, ^{1.7}H), 3.03-2.98 (m, ^{0.5}H), 2.91 (s, ^{1.3}H), 2.51 (brs, ³H), 2.00-1.87 (m, ¹H), 1.10 (two doublets, J=6.3, 6.3 Hz, ³H), 0.93-0.81 (m, ⁶H).

Compound 12d was synthesized from (2S,3S)-2-amino-3-hydroxy-N-isobutyl-N-methyl-butanamide by reductive amination with aldehyde 11 following a similar procedure described for the synthesis of compound 12a (yield 30%). ¹H NMR (300 MHz, CDCl₃): δ 7.33-506 7.10 (m, ⁵H), 4.80-4.64 (m, ¹H), 3.95-3.78 (m, ²H), 3.43 (dd, J=11.8, 4.4 Hz, ¹H), 3.35-3.09 (m, ²H), 3.06-2.96 (m, ^{2.5}H), 2.91 (s, ^{1.5}H), 2.84 (br, ²H), 2.72-2.59 (m, ¹H), 2.48-2.33 (m, ¹H), 2.01-1.87 (m, ¹H), 1.39 (s, ⁹H), 1.10 and 1.06 (two doublets, J=6.4, 6.4 Hz, ³H), 0.90 and 0.86 (two doublets, J=6.7, 6.3 Hz, ⁶H).

### Synthesis of Inhibitor 7

Inhibitor 7 was synthesized from compound 12d by boc removal using trifluoroacetic acid followed by coupling of the resulting amine with the known acid 13 using EDC, HOBt, and iPr2NEt following a similar procedure described for the synthesis of inhibitor 4 (yield 61%, over two steps). ¹H NMR (400 MHz, CDCl₃): δ 8.21 and 8.17 (two singlets, ¹H), 8.07-7.92 (m, ²H), 7.45-7.30 (m, ⁴H), 7.30-7.15 (m, ⁶H), 7.07 (t, J=7.8 Hz, ¹H), 5.40-5.26 (m, ¹H), 4.34-4.19 (m, ¹H), 3.97-3.82 (m, ¹H), 3.55 and 3.53 (two doublets, J=4.2, 4.2 Hz, ¹1H), 3.33 (s, ³H), 3.23-3.03 (m, ^{2.5}H), 3.01 (s, ²H), 2.95-2.87 (m, ¹H), 2.85 and 2.84 (two singlets, ³H), 2.77 (s, ¹H), 2.73-2.57 (m, ^{2.5}H), 1.96-1.78 (m, ¹H), 1.60 (d, J=6.9 Hz, ³H), 1.13 and 1.10 (two doublets, J=6.4, 6.5 Hz, ³H), 0.89 and 0.84 (two doublets, J=6.6, 6.5 Hz, 2.7H), 0.79 and 0.75 (two doublets, J=6.7, 6.7 Hz, 3.3H). HRMS-ESI (m/z): [M+H]+ calcd for C₃₆H₅₀N₅O₆S, 680.3482; found, 680.3478. Synthesis of (2S,3S)-2-[(tert-Butoxycarbonyl)amino]-3-methoxy-butanoic Acid (15). To a solution of (2S,3S)-methyl 2-[(tert-butoxycarbonyl)amino]-3-hydroxybutanoate (14) (2.14 mmol, 0.5 g) in CH₃CN (21 mL) were added Ag₂O (10.7 mmol, 2.48 g) and MeI (21.4 mmol, 1.3 mL) at 23° C., and the resulting reaction mixture was stirred for 8 days at 23° C. The reaction mixture was filtered and concentrated under reduced pressure. The residue was purified by silica gel column chromatography (20-25% EtOAc/hexanes) to furnish (2S,3S)-methyl 2-[(tert-butoxycarbonyl)amino]-3-methoxybutanoate in 55% yield (0.29 g). ¹H NMR (400 MHz, CDCl₃): δ 5.27 (d, J=9.3 Hz, ¹H), 4.41 (dd, J=8.6, 3.6 Hz, ¹H), 3.74 (s, ³H), 3.66-3.55 (m, ¹H), 3.34 (s, ¹H), 1.43 (s, ¹H), 1.18 (d, J=6.4 Hz, ³H).

To a solution of (2S,3S)-methyl 2-[(tert-butoxycarbonyl)amino]-3-methoxybutanoate (0.86 mmol, 0.213 g) in THF (4 mL) and H₂O (2 mL) was added LiOH.H₂O (5.16 mmol, 0.216 g) at 23° C. The resulting reaction mixture was stirred for 5 hr at 23° C. The reaction mixture was diluted with H₂O and diethyl ether. The organic layer was separated, and the aqueous layer was carefully acidified with 2N HCl and extracted with ethyl acetate. The combined extracts were dried over anhydrous Na₂SO4, filtered, and concentrated under reduced pressure to provide crude (2S,3S)-2-[(tert-butoxycarbonyl)amino]-3-methoxybutanoic acid (15). This crude acid was used in the coupling reaction without any further purification.

### Synthesis of Compound 12e

The synthesis of tert-butyl [(2S,3S)-1-(isobutylamino)-3-methoxy-1-oxobutan-2-yl]carbamate has been carried out by coupling of the above crude acid 15 with isobutyl amine using EDC, HOBt, and iPr2NEt following the procedure described for the synthesis of (S)-tert-butyl [1-(isobutylamino)-1-oxobutan-2-yl]carbamate (yield 74%, over two steps). ¹H NMR (300 MHz, CDCl₃): δ 6.22 (brs, ¹H), 5.15 (brs, ¹H), 4.10 (dd, J=7.7, 6.8 Hz, ¹H), 3.59 (p, J=6.4 Hz, ¹H), 3.34 (s, ³H), 3.21-3.10 (m, ¹H), 3.09-2.98 (m, 1¹H), 1.77 (hept, J=6.7 Hz, ¹H), 1.44 (s, ⁹H), 1.18 (d, J=6.3 Hz, ³H), 0.91 (d, J=6.8 Hz, ⁶H).

To a solution of tert-butyl [(2S,3S)-1-(isobutylamino)-3-methoxy-1-oxobutan-2-yl]-carbamate (0.624 mmol, 0.18 g) in CH₂Cl₂ (6 mL) was added trifluoroacetic acid (2 mL) at 0° C., and the resulting mixture was stirred for 1.5 hr at 23° C. Trifluoroacetic acid and CH₂Cl₂ were removed under reduced pressure, and the resulting residue was diluted with EtOAc and basified with saturated aqueous NaHCO₃ solution. The organic layer was separated, and the aqueous layer was extracted several times with EtOAc. The combined organic layers were dried over anhydrous Na₂SO₄, filtered, and concentrated under reduced pressure to obtain crude (2S,3S)-2-amino-N-isobutyl-3-methoxybutanamide in 88% yield.

To a solution of the above crude amine (0.55 mmol, 0.104 g) and (S)-tert-butyl (1-oxo-3-phenylpropan-2-yl)carbamate 11 [prepared from the corresponding Weinreb amide (0.55 mmol) following a similar literature procedure]7 in CH₂Cl₂ (6 mL), Na(OAc)₃BH (0.825 mmol, 0.175 g) was added at 0° C. The resulting mixture was stirred for 1 hr at 0° C. and 15 hr at 23° C. The reaction mixture was quenched with saturated aqueous NaHCO₃ solution and extracted with CH₂Cl₂. The combined extracts were dried over anhydrous Na₂SO₄, filtered, and concentrated under reduced pressure. The residue was purified by silica gel column chromatography (20-45% EtOAc/hexanes) to furnish the corresponding adduct 12e in 67% yield (0.155 g). 1H NMR (400 MHz, CDCl₃): δ 7.35 (brs, ¹H), 7.29-7.21 (m, ²H), 7.21-7.10 (m, ³H), 4.64 (d, J=8.2 Hz, ¹H), 3.94 (brs, ¹H), 3.77-3.68 (m, ¹H), 3.29 (s, ³H), 3.27 (d, J=4.0 Hz, ¹H), 3.00 (t, J=6.4 Hz, ²H), 2.82 (dd, J=12.9, 5.7 Hz, ¹H), 2.69 (dd, J=13.6, 7.7 Hz, ¹H), 2.58-2.46 (m, ²H), 1.68 (hept, J=6.7 Hz, ¹H), 1.39 (s, ⁹H), 1.00 (d, J=6.4 Hz, ³H), 0.83 (d, J=6.6 Hz, ⁶H).

### Synthesis of Inhibitor 8

To a solution of 12e (0.073 mmol, 30.8 mg) in CH₂Cl₂ (3 mL) was added trifluoroacetic acid (1 mL) at 0° C. The resulting reaction mixture was warmed to 23° C. and stirred for 1.5 hr at 23° C. The reaction mixture was concentrated under reduced pressure, and the resulting residue was used in the next step without any further purification. To a solution of the above residue in CH₂Cl₂ (5 mL) were added 593 iPr2NEt (0.1 mL), HOBt.H₂O (0.14 mmol, 18.9 mg), acid 13 (0.07 mmol, 26.3 mg), and EDC.HCl (0.14 mmol, 26.8 mg) simultaneously at 23° C. The resulting reaction mixture was stirred for 17 hr at 23° C. The reaction mixture was quenched with saturated aqueous NaHCO3 solution and extracted with CH₂Cl₂. The combined extracts were dried over anhydrous Na₂SO₄, filtered, and concentrated under reduced pressure. The residue was purified by silica gel column chromatography (2% MeOH/CH₂Cl₂) to obtain the inhibitor 8 in 67% yield (32.1 mg). ¹H NMR (400 MHz, CDCl₃): δ 8.19 (s, ¹H), 8.02 (s, ¹H), 7.96 (s, ¹H), 7.42-7.29 (m, ⁵H), 7.28-7.25 (m, ²H), 7.22 (d, J=7.4 Hz, ³H), 7.14-7.02 (m, ³H), 5.32 (p, J=7.2 Hz, ¹H), 4.48-4.36 (m, ¹H), 3.73-3.61 (m, ¹H), 3.33 (s, ³H), 3.30 (s, ³H), 3.27 (d, J=4.2 Hz, ¹H), 3.04-2.94 (m, ³H), 2.90-2.80 (m, ⁴H), 2.76-2.65 (m, ²H), 1.69-1.61 (m, ¹H), 1.61 (d, J=6.9 Hz, ³H), 1.04 (d, J=6.4 Hz, ³H), 0.80 (d, J=6.4 Hz, ⁶H). ¹³C NMR (125 MHz, CDCl₃): δ 171.84, 165.41, 164.41, 142.87, 142.20, 137.42, 135.67, 135.64, 129.11, 128.60, 128.57, 127.80, 127.73, 127.38, 126.63, 126.18, 123.41, 65.95, 56.52, 51.99, 51.19, 49.53, 46.33, 38.73, 37.83, 35.43, 28.32, 21.63, 20.00, 14.62. HRMS-ESI (m/z): [M+H]+ calcd for C₃₆H₅₀N₅O₆S, 680.3482; found, 680.3488.

### Synthesis of tert-Butyl {(2R,3S)-3-[(tert-Butyldimethylsilyl)oxy]-1-(isobutylamino)butan-2-yl}carbamate (16)

To a solution of (2S,3S)-methyl 2-[(tert-butoxycarbonyl)amino]-3-hydroxybutanoate (14) (2.2 mmol, 0.513 g) in CH₂Cl₂ (11 mL) were added Et₃N (3.3 mmol, 0.46 mL) and TBSOTf (2.86 mmol, 0.66 mL) at 0° C. The resulting reaction mixture was warmed to 23° C. and stirred for 2 hr at the same temperature. The reaction mixture was diluted with H₂O and CH₂Cl₂. The organic layer was separated, and the aqueous layer was extracted with CH₂Cl₂. The combined extracts were dried over anhydrous Na₂SO₄, filtered, and concentrated under reduced pressure. The residue was purified by silica gel column chromatography (10% EtOAc/hexanes) to obtain (2S,3S)-methyl 2-[(tert-butoxycarbonyl)-amino]-3-[(tert-butyldimethylsilyl)oxy]butanoate in 88% yield (0.67 g). ¹H NMR (400 MHz, CDCl₃): δ 5.25 (d, J=8.4 Hz, ¹H), 4.16 (dd, J=8.3, 3.7 Hz, ¹H), 4.05-3.96 (m, ¹H), 3.66 (s, ³H), 1.36 (s, ⁹H), 1.16 (d, J=6.3 Hz, ³H), 0.79 (s, ⁹H), −0.03 (s, ⁶H).

To a solution of (2S,3S)-methyl 2-[(tert-butoxycarbonyl)amino]-3-[(tert-butyldimethyl-silyl)oxy]butanoate (0.288 mmol, 0.1 g) in Et²O (3 mL) was added DiBAL-H (0.63 mmol, 0.63 mL, 1 M solution in CH₂Cl₂) at −78° C., and the resulting mixture was stirred for 1.5 hr at −78° C. The reaction mixture was quenched with 0.5 mL of EtOAc and diluted with Et₂O and saturated aqueous sodium potassium tartrate. After it was stirred for 0.5 hr, the organic layer was separated, and the aqueous layer was extracted with Et₂O. The combine extracts were washed with brine, dried over anhydrous Na₂SO₄, filtered, and concentrated under reduced pressure. The resulting residue was used in the next step without any further purification.

To a solution of the above aldehyde in CH₂Cl₂ (6 mL) were added isobutyl amine (0.864 mmol, 0.086 mL) and Na(OAc)₃BH (0.576 mmol, 0.122 g) at 0° C., and the resulting reaction mixture was stirred for 1.5 hr at 0° C. and 15 hr at 23° C. The reaction mixture was quenched with saturated aqueous NaHCO3 solution and extracted with CH₂Cl₂. The combined extracts were dried over anhydrous Na₂SO₄, filtered, and concentrated under reduced pressure. The resulting residue was purified by silica gel column chromatography [3% (5% NH₃/MeOH)/CH₂Cl₂] to obtain (tert-butyl {(2R,3S)-3-[(tert-butyldimethylsilyl)oxy]-1-(isobutylamino)butan-2-yl}-carbamate (16) in 75% yield (80.7 mg). ¹H NMR (400 MHz, CDCl₃): δ 5.36 (d, J=7.9 Hz, ¹H), 4.11-3.98 (m, ¹H), 3.57-3.42 (m, ¹H), 2.90 (dd, J=12.2, 5.3 Hz, ¹H), 2.62 (dd, J=12.2, 4.3 Hz, ¹H), 2.44-2.30 (m, ²H), 1.72 (hept, J=6.7 Hz, ¹H), 1.43 (s, ⁹H), 1.14 (d, J=6.4 Hz, ³H), 0.92-0.84 (m, ¹⁵H), 0.04 (s, ³H), 0.03 (s, ³H).

### Synthesis of Compound 17

Compound 17 was synthesized by Boc removal of tert-butyl {(2R,3S)-3-[(tert-butyldimethylsilyl)oxy]-1-(isobutylamino)butan-2-yl}carbamate (16) using trifluoroacetic acid followed by reductive amination with known aldehyde 11 following the procedure described for the synthesis of 12e. ¹H NMR (400 MHz, CDCl₃): δ 7.31-7.23 (m, ²H), 7.23-7.15 (m, ³H), 5.20 (br, ¹H), 3.94-3.75 (m, ²H), 2.95-2.81 (m, ¹H), 2.79-2.67 (m, ²H), 2.58 (dd, J=12.4, 5.0 Hz, ²H), 2.50-2.43 (m, ²H), 2.38 (qd, J=11.5, 6.9 Hz, ²H), 1.74 (hept, J=6.7 Hz, ¹H), 1.40 (s, ⁹H), 1.10 (d, J=6.3 Hz, ³H), 0.90 (d, J=6.6 Hz, ⁶H), 0.87 (s, ⁹H), 0.04 (s, ³H), 0.02 (s, ³H).

### Synthesis of Inhibitor 9

Boc removal of compound 17 using trifluoroacetic acid followed by coupling of the resulting amine with known acid 13 using EDC, HOBt, and iPr2NEt following the procedure described for the synthesis of inhibitor 8 afforded the corresponding amide in 70% yield. ¹H NMR (400 MHz, CDCl₃): δ 8.20 (s, ¹H), 8.00 (s, ¹H), 7.94 (d, J=7.8 Hz, ¹H), 7.92 (s, ¹H), 7.66 (d, J=7.6 Hz, ¹H), 7.40 (d, J=7.5 Hz, ²H), 7.33 (t, J=7.5 Hz, ²H), 7.29-7.19 (m, ⁵H), 7.20-7.12 (m, ¹H), 5.29 (p, J=7.1 Hz, ¹H), 4.20-4.06 (m, ¹H), 4.06-3.93 (m, ¹H), 3.28 (s, ³H), 3.20 (dd, J=12.4, 8.2 Hz, ¹H), 3.10-2.98 (m, ²H), 2.86 (dd, J=13.6, 6.4 Hz, ¹H), 2.82-2.76 (m, ⁴H), 2.76-2.67 (m, ³H), 2.60 (dd, J=12.4, 7.1 Hz, ¹H), 1.81 (hept, J=6.8 Hz, ¹H), 1.59 (d, J=7.0 Hz, ³H), 1.11 (d, J=6.4 Hz, ³H), 0.86 (s, ⁹H), 0.75 (d, J=6.7 Hz, ³H), 0.75 (d, J=6.7 Hz, ³H), 0.07 (s, ³H), 0.05 (s, ³H).

To a solution of the above amide (0.104 mmol, 79.7 mg) in THF (10 mL) was added TBAF (1.25 mmol, 1.25 mL, 1 M solution in THF) at 0° C., and the resulting reaction mixture was stirred for 18 hr at 23° C. The solvent was removed under reduced pressure, and the resulting residue was purified by silica gel column chromatography [3-5% (5% NH₃/MeOH)/CH₂Cl₂] to obtain inhibitor 9 in 65% (43.9 mg) yield. ¹H NMR (400 MHz, CDCl₃): δ 8.24 (s, ¹H), 8.01-7.93 (m, ¹H), 7.40 (d, J=7.3 Hz, ²H), 7.37-7.24 (m, ⁷H), 7.24-7.16 (m, ³H), 5.31 (p, J=7.3 Hz, ¹H), 4.37-4.25 (m, ¹H), 3.93-3.83 (m, ¹H), 3.31 (s, ³H), 3.04 (dd, J=13.5, 5.8 Hz, ¹H), 2.81 (s, ³H), 2.80-2.67 (m, ⁵H), 2.44-2.27 (m, ³H), 1.70-1.54 (m, ⁴H), 1.13 (d, J=6.8 Hz, ³H), 0.81 (d, J=6.5 Hz, ⁶H). ¹³C NMR (100 MHz, CDCl₃): δ 165.78, 164.64, 143.13, 142.13, 137.87, 135.80, 129.24, 128.63, 128.53, 128.16, 127.46, 127.37, 126.53, 126.34, 123.78, 68.48, 60.61, 57.62, 51.94, 49.74, 49.48, 49.30, 38.67, 37.88, 35.46, 27.73, 21.78, 20.41, 20.38, 20.34. HRMS-ESI (m/z): [M+H]+ calcd for C₃₅H₅₀N₅O₅S, 652.3533; found, 652.3543.

### Synthesis of Inhibitor 18

Inhibitor 18 was synthesized from compound 12b by Boc removal using trifluoroacetic acid followed by coupling of the resulting amine with known acid 23 using EDC, HOBt, and iPr2NEt following a similar procedure described for the synthesis of inhibitor 4 (yield 68%, over two steps). ¹H NMR (400 MHz, CDCl₃): δ 7.85 (s, ¹H), 7.47 (s, ¹H), 7.38-7.28 (m, ³H), 7.24-7.19 (m, ²H), 6.84 (s, ¹H), 6.47 (d, J=8.0 Hz, ¹H), 4.62-4.36 (m, ³H), 4.02 (p, J=6.0 Hz, ¹H), 3.90-3.81 (m, ²H), 3.47 (s, ³H), 3.12 (d, J=5.0 Hz, ¹H), 3.10-2.93 (m, ⁴H), 2.84 (dd, J=12.2, 4.7 Hz, ¹H), 2.77 (dd, J=11.1, 6.8 Hz, ¹H), 2.72 (q, J=7.5 Hz, ²H), 1.76-1.65 (m, ¹H), 1.30 (t, J=7.5 Hz, ³H), 1.15 (d, J=6.4 Hz, ³H), 0.85 (d, J=6.7 Hz, ³H), 0.84 (d, J=6.6 Hz, ³H). ¹³C NMR (100 MHz, CDCl₃): δ 172.75, 167.90, 137.52, 133.84, 130.72, 129.28, 128.65, 127.72, 127.23, 126.75, 125.78, 120.26, 118.03, 117.60, 68.32, 67.48, 56.77, 52.30, 51.39, 46.41, 43.60, 39.66, 39.03, 28.42, 20.11, 18.95, 17.93, 14.25. HRMS-ESI (m/z): [M+H]+ calcd for C³¹H₄₄N₅O₅S, 598.3063; found, 598.3060.

### Synthesis of Compound 25

To a solution of 24 (0.43 mmol, 0.145 g) in DMF (4 mL) was added NaH (1.72 mmol, 68.8 mg, 60% NaH in mineral oil) at 23° C., and the resulting mixture was stirred for 15 min at the same temperature. Iodo methane (1.72 mmol, 0.11 mL) was added to the reaction mixture, and stifling was continued for further 2.5 hr at 23° C. The reaction mixture was quenched with methanol and then diluted with EtOAc. The resulting solution was washed with H₂O and brine, dried over anhydrous Na₂SO₄, filtered, and concentrated under reduced pressure. The resulting residue was purified by silica gel column chromatography (30% EtOAc/hexanes) to obtain the corresponding α,α-dimethylated adduct in 51% (76.8 mg) yield. ¹H NMR (400 MHz, CDCl₃): δ 8.18 (d, J=1.0 Hz, ¹H), 7.74 (s, ¹H), 6.81 (s, ¹H), 4.14 (s, ²H), 3.93 (s, ³H), 3.55 (s, ³H), 2.76 (q, J=7.5 Hz, ²H), 1.57 (s, ⁶H), 1.32 (t, J=7.5 Hz, ³H). A mixture of the above ester (0.219 mmol, 76.7 mg) and NaOH (20 mmol, 10 mL, 2N NaOH) in EtOH (10 mL) and THF (10 mL) was stirred for 2.5 days at 23° C. The solvent was removed under reduced pressure, and the resulting mixture was diluted with H₂O and diethyl ether. The organic layer was separated, and the aqueous layer was acidified with aqueous 1N HCl and extracted with ethyl acetate. The combined extracts were dried over anhydrous Na₂SO₄, filtered, and concentrated under reduced pressure to furnish the corresponding crude acid (25) in 72% yield (53.2 mg), which was used directly in the coupling reaction without any further purification. LRMS-ESI (m/z): [M+Na]+, 359.19

### Synthesis of 1-(Benzyloxymethyl)cyclopropanesulfonyl Chloride (27)

Solutions of BuLi [17.2 mmol, 6.9 mL (2.5 M in hexanes) in 15 mL of THF] and butyl 3-chloro-1-propanesulfonate (26) (16.3 mmol, 3.5 g in 15 mL of THF) were added at the same time via cannula to an oven-dried flask containing THF (100 mL) at −78° C., and the resulting mixture was stirred for 5-10 min at −78° C. and 30 min at 0° C. The reaction mixture was cooled back to −78° C., and a solution of BuLi [19.6 mmol, 7.8 mL (2.5 M in hexanes)] was added to this mixture. After it was stirred for 15 min at −78° C., BOMCl (19.6 mmol, 2.7 mL) was added to the reaction flask, and stifling was continued for further 2 hr at −78° C. and 3r hr at 23° C. The reaction mixture was quenched with H₂O, and THF was removed under reduced pressure. The resulting mixture was diluted with CH₂Cl₂ and H₂O. The organic layer was separated, and the aqueous layer was extracted with CH₂Cl₂. The combined extracts were dried over anhydrous Na₂SO₄, filtered, and concentrated under reduced pressure. The residue was purified by silica gel column chromatography (8-12% EtOAc/hexanes) to furnish butyl 1-(benzyloxymethyl)-cyclopropanesulfonate in 80% yield (3.9 g). ¹H NMR (300 MHz, CDCl₃): δ 7.40-7.25 (m, ⁵H), 4.55 (s, ²H), 4.23 (t, J=6.6 Hz, ²H), 3.79 (s, ²H), 1.73-1.58 (m, ²H), 1.48 (ABq, J=6.9, 5.0 Hz, ²H), 1.44-1.30 (m, ²H), 1.11 (ABq, J=7.0, 5.1 Hz, ²H), 0.90 (t, J=7.4 Hz, ³H). ¹³C NMR (75 MHz, CDCl₃): δ 137.41, 128.33, 127.78, 127.61, 73.09, 70.66, 69.84, 37.81, 31.04, 18.55, 13.41, 10.72.

To a mixture of butyl 1-(benzyloxymethyl)cyclopropanesulfonate (13 mmol, 3.88 g) in DME (40 mL) and H₂O (40 mL), KSCN (13.65 mmol, 1.33 g) was added at 23° C., and the resulting reaction mixture was refluxed for 15 h. The reaction mixture was cooled to 23° C. and diluted with H₂O and ethyl acetate. The organic layer was separated, and the aqueous layer was concentrated under reduced pressure to provide the crude potassium 1-(benzyloxymethyl)-cyclopropanesulfonate, which was used directly in the next step without additional purification. A mixture of potassium 1-(benzyloxymethyl)cyclopropanesulfonate in SOCl₂ (35 mL) and DMF (3.5 mL) was refluxed for 1.5 h, and excess SOCl₂ was removed under reduced pressure. Water was added carefully to the resulting mixture and extracted with ethyl acetate. The combined extracts were dried over anhydrous Na₂SO₄, filtered, and concentrated under reduced pressure. The residue was purified by silica gel column chromatography (5-10% EtOAc/hexanes) to obtain 1-(benzyloxymethyl)cyclopropanesulfonyl chloride 27 in 91% yield (3.1 g) (over two steps). ¹H NMR (300 MHz, CDCl₃): δ 7.46-7.28 (m, ⁵H), 4.63 (s, ²H), 4.01 (s, ²H), 1.85-1.77 (m, ²H), 1.46-1.37 (m, ²H). ¹³C NMR (75 MHz, CDCl₃): δ 136.99, 128.41, 127.89, 127.61, 73.29, 68.38, 52.39, 14.03.

### Synthesis of Sulfonamide 29

To a mixture of amine 28 (3.66 mmol, 0.8 g), pyridine (11 mmol, 0.89 mL), and DMAP (0.73 mmol, 89.2 mg) in CH₂Cl₂ at 0° C. was added a solution of 1-(benzyloxymethyl)-cyclopropanesulfonyl chloride (27) (3.84 mmol, 1 g in 5 mL of CH₂Cl₂), and the resulting mixture was stirred for 44 hr at 23° C. The reaction mixture was diluted with CH₂Cl₂, washed with aqueous 1N HCl and brine, and dried over anhydrous Na₂SO₄. Dichloromethane solution was filtered and concentrated under reduced pressure. The residue was purified by silica gel column chromatography (20-30% EtOAc/hexanes) to afford the corresponding sulfonamide 29 in 74% yield (1.2 g). ¹H NMR (400 MHz, CDCl₃): δ 9.38 (s, ¹H), 8.25 (s, ¹H), 7.87 (s, ¹H), 7.49 (d, J=7.1 Hz, ²H), 7.42 (t, J=7.4 Hz, ²H), 7.39-7.33 (m, ¹H), 6.79 (s, ¹H), 6.69 (s, ¹H), 4.73 (s, ²H), 3.91 (s, ³H), 3.90 (s, ²H), 2.75 (q, J=7.5 Hz, ²H), 1.30 (t, J=7.5 Hz, ³H), 1.11-0.99 (m, ²H), 0.77-0.65 (m, ²H). ¹³C NMR (100 MHz, CDCl³): δ 167.62, 136.35, 135.01, 128.91, 128.63, 128.44, 122.14, 121.20, 120.85, 120.76, 120.61, 120.36, 74.15, 73.10, 51.84, 38.91, 18.09, 14.25, 10.25.

### Synthesis of 7,6,5-Tricyclic Indole Derivative 30

To a solution of sulfonamide 29 (2.7 mmol, 1.19 g) in MeOH (75 mL) and AcOH (25 mL), 10% Pd/C (0.2 g) was added under argon. The argon balloon was now replaced with a H₂ balloon, and the resulting mixture was stirred for 16 hr at 23° C. The reaction mixture was filtered through Celite and washed with MeOH. The solvent was removed under reduced pressure, and the resulting residue was diluted with toluene and concentrated under reduced pressure to furnish the corresponding alcohol in 95% yield.

To a mixture of the above alcohol (0.6 mmol, 0.21 g) and Et₃N (0.9 mmol, 0.12 mL) in CH₂Cl₂ (25 mL) at 0° C. was added methanesulfonyl chloride (0.63 mmol, 0.049 mL), and the resulting mixture was stirred for 2 hr at 23° C. The reaction mixture was diluted with CH₂Cl₂, washed with aqueous 1N HCl and brine, and dried over anhydrous Na₂SO₄. Dichloromethane solution was filtered and concentrated under reduced pressure. The residue was purified by silica gel column chromatography (5-15% diethyl ether/CH₂Cl₂) to afford the corresponding mesylate in 46% (70% BRSM) yield (0.12 g, 69 mg of alcohol was recovered). ¹H NMR (400 MHz, CDCl₃): δ 9.24 (s, ¹H), 8.26 (s, ¹H), 7.87 (d, J=1.0 Hz, ¹H), 7.41 (s, ¹H), 7.07 (s, ¹H), 4.58 (s, ²H), 3.93 (s, ³H), 3.16 (s, ³H), 2.79 (q, J=7.5 Hz, ²H), 1.38-1.28 (m, ⁵H), 1.06-0.97 (m, ²H).

To a solution of the above mesylate (0.69 mmol, 0.297 g) in DMF (30 mL) was added NaH (2.76 mmol, 0.11 g, 60% dispersion in mineral oil) at 23° C. After the mixture was stirred for 3 hr at 23° C., iodo methane (3.5 mmol, 0.22 mL) was added to the flask, and stifling was continued for further 1 hr at 23° C. The reaction mixture was carefully quenched with MeOH, diluted with ethyl acetate, and washed with aqueous 1N HCl. The ethyl acetate solution was filtered and concentrated under reduced pressure. The resulting residue was purified by silica gel column chromatography (35-40% EtOAc/hexanes) to afford the corresponding 7,6,5-tricyclic indole derivative in 87% yield (0.21 g). ¹H NMR (400 MHz, CDCl₃): δ 8.26 (d, J=1.1 Hz, ¹H), 7.83 (d, J=1.0 Hz, ¹H), 6.78 (s, ¹H), 4.32 (s, ²H), 3.93 (s, ³H), 3.48 (s, ³H), 2.77 (q, J=7.5 Hz, ²H), 1.53 (t, J=6.6 Hz, ²H), 1.31 (t, J=7.5 Hz, ³H), 1.03-0.95 (m, ²H). A mixture of the above 7,6,5-tricyclic indole derivative (0.2 mmol, 69.7 mg) and NaOH (20 mmol, 0.8 g in 10 mL of H₂O) in EtOH (5 mL) and THF (10 mL) was stirred for 4 days at 23° C. The solvent was removed under reduced pressure, and the resulting mixture was diluted with H₂O and diethyl ether. The organic layer was separated, and the aqueous layer was acidified with aqueous 1N HCl and extracted with ethyl acetate. The combined extracts were dried over anhydrous Na₂SO₄, filtered, and concentrated under reduced pressure to furnish the corresponding crude acid (30) in 75% yield (50 mg), which was used directly in the coupling reaction without any further purification. LRMS-ESI (m/z): [M+Na]+, 357.26.

### Synthesis of (2R,3S)-Ethyl 3-Hydroxy-2-{[(4-nitrophenyl)sulfonyl]-oxy}hexanoate (32)

To a solution of (E)-ethyl hex-2-enoate (31) (12.6 mmol, 1.79 g) in tBuOH (30 mL) and H₂O (30 mL) were added AD-mixα (17.6 g) and MeSO₂NH₂ (15.1 mmol, 1.44 g) at −1° C., and the resulting reaction mixture was stirred for 7 days at −1° C. The reaction mixture was quenched with saturated aqueous Na₂S₂O₃ solution and extracted with ethyl acetate. The combined extracts were dried over anhydrous Na₂SO₄, filtered, and concentrated under reduced pressure. The residue was purified by silica gel column chromatography (20-35% EtOAc/hexanes) to furnish (2R,3S)-ethyl 2,3-dihydroxyhexanoate in 60% yield (1.34 g). ¹H NMR (300 MHz, CDCl₃): δ 4.29 (q, J=7.2 Hz, ²H), 4.10-4.04 (m, ¹H), 3.96-3.84 (m, ¹H), 3.07 (d, J=5.2 Hz, ¹H), 1.90 (d, J=7.7 Hz, ¹H), 1.68-1.38 (m, ⁴H), 1.32 (t, J=7.1 Hz, ³H), 0.96 (t, J=7.2 Hz, ³H). To a solution of (2R,3S)-ethyl 2,3-dihydroxyhexanoate (6.7 mmol, 1.18 g) in CH₂Cl₂ (30 mL) were added Et3N (10 mmol, 1.39 mL) and 4-nitrobenzenesulfonyl chloride (6.7 mmol, 1.48 g) at 0° C. The resulting reaction mixture was stirred for 24 hr at 23° C. The reaction mixture was diluted with H₂O and CH₂Cl₂. The organic layer was separated, and the aqueous layer was extracted with CH₂Cl₂. The combined extracts were dried over anhydrous Na₂SO₄, filtered, and concentrated under reduced pressure. The residue was purified by silica gel column chromatography (10-25% EtOAc/hexanes) to obtain (2R,3S)-ethyl 3-hydroxy-2-{[(4-nitrophenyl)sulfonyl]oxy}-hexanoate (32) in 54% yield (1.3 g). ¹H NMR (400 MHz, CDCl₃): δ 8.38 (d, J=8.8 Hz, ²H), 8.16 (d, J=8.9 Hz, ²H), 4.96 (d, J=2.9874 Hz, ¹H), 4.15 (q, J=7.1 Hz, ²H), 4.12-4.04 (m, ¹H), 2.07 (brs, ¹H), 1.62-1.44 (m, ³H), 1.43-1.30 (m, ¹H), 1.21 (t, J=7.1 Hz, ³H), 0.92 (t, J=7.0 Hz, ³H). ¹³C NMR (100 MHz, CDCl₃): δ 166.74, 150.75, 141.85, 129.45, 124.18, 80.97, 71.23, 62.30, 34.98, 18.47, 13.89, 13.63. f

### Synthesis of (2S,3S)-Ethyl 2-[(tert-Butoxycarbonyl)amino]-3-hydroxyhexanoate (33)

To a solution of (2R,3S)-ethyl 3-hydroxy-2-{[(4-nitrophenyl)sulfonyl]oxy}hexanoate (32) (3.6 mmol, 1.3 g) in DMF (10 mL), NaN₃ (5.76 mmol, 0.374 g) was added at 23° C., and the resulting mixture was heated at 55° C. for 15 h. The reaction mixture was cooled to 23° C. and diluted with ethyl acetate, and the resulting solution was washed with H₂O, dried over anhydrous Na₂SO₄, filtered, and concentrated under reduced pressure. The residue was purified by silica gel column chromatography (15% EtOAc/hexanes) to obtain (2S,3S)-ethyl 2-azido-3-hydroxyhexanoate in 83% yield (0.6 g). ¹H NMR (400 MHz, CDCl₃): δ 4.29 (qd, J=7.2, 1.3 Hz, ²H), 3.94 (s, ²H), 2.32 (brs, ¹H), 1.59-1.45 (m, ³H), 1.44-1.37 (m, ¹H), 1.33 (t, J=7.2 Hz, ³H), 0.94 (t, J=6.9 Hz, ³H). ¹³C NMR (100 MHz, CDCl₃): δ 168.92, 71.56, 66.15, 61.99, 35.03, 18.52, 14.06, 13.74.

To a mixture of (2S,3S)-ethyl 2-azido-3-hydroxyhexanoate (2.98 mmol, 0.6 g) and (Boc)2O (4.47 mmol, 0.975 g) in EtOH (15 mL) was added 10% Pd—C (0.15 g) at 23° C. under argon. The argon balloon was replaced with a H₂ balloon, and the reaction mixture was stirred for 15 hr under H₂ atmosphere. The reaction mixture was filtered through Celite, washed with ethyl acetate, and concentrated under reduced pressure. The residue was purified by silica gel column chromatography (10-25% EtOAc/hexanes) to obtain (2S,3S)-ethyl 2-[(tert-butoxycarbonyl)amino]-3-hydroxyhexanoate (33) in 96% yield (0.79 g). ¹H NMR (300 MHz, CDCl₃): δ 5.52 (d, J=7.2 Hz, ¹H), 4.42-4.28 (m, 1H), 4.27-4.13 (m, ²H), 3.94-3.80 (m, ¹H), 3.03 (brs, ¹H), 1.59-1.30 (m, ¹³H), 1.26 (t, J=7.1 Hz, ³H), 0.89 (t, J=6.9 Hz, ³H). ¹³C NMR (75 MHz, CDCl₃): δ 170.68, 155.98, 80.20, 72.67, 61.48, 58.34, 35.29, 28.18, 18.86, 14.06, 13.82. LRMS-ESI (m/z): [M+Na]+, 298.26.

### Synthesis of tert-Butyl [(2S,3S)-3-Hydroxy-1-(isobutylamino)-1-oxohexan-2-yl]carbamate (34a)

To a solution of (2S,3S)-ethyl 2-[(tert-butoxycarbonyl)amino]-3-hydroxyhexanoate (33) (0.67 mmol, 0.18 g) in THF (6 mL) and H₂O (3 mL) was added LiOH.H₂O (3.45 mmol, 0.145 g). The resulting reaction mixture was stirred for 12 hr at 23° C. The reaction mixture was diluted with H₂O and ethyl acetate. The organic layer was separated, and the aqueous layer was acidified with 1N HCl and extracted with ethyl acetate. The combined extracts were dried over anhydrous Na₂SO₄, filtered, and concentrated under reduced pressure. The residue was used in the coupling reaction without any further purification. The synthesis of tert-butyl [(2S,3S)-3-hydroxy-1-(isobutylamino)-1-oxohexan-2-yl]carbamate (34a) has been carried out by coupling of (2S,3S)-2-[(tert-butoxycarbonyl)amino]-3-hydroxyhexanoic acid with isobutyl amine using EDC, HOBt, and iPr2NEt following a similar procedure described for the synthesis of (S)-tert-butyl [1-(isobutylamino)-1-oxobutan-2-yl]carbamate (yield 60%, over two steps). ¹H NMR (400 MHz, CDCl₃): δ 6.51 (brs, ¹H), 5.62 (d, J=7.2 Hz, ¹H), 3.98-3.82 (m, ²H), 3.69 (brs, ¹H), 3.21-3.08 (m, ¹H), 3.06-2.92 (m, ¹H), 1.77 (hept, J=6.7 Hz, ¹H), 1.63-1.46 (m, ³H), 1.44 (s, ⁹H), 1.41-1.32 (m, ¹H), 0.97-0.83 (m, ⁹H).

### Synthesis of Compound 35a

Compound 35a was synthesized from tert-butyl [(2S,3S)-3-hydroxy-1-(isobutylamino)-1-oxohexan-2-yl]carbamate (34a) by Boc removal using trifluoroacetic acid followed by reductive amination of the resulting amine with aldehyde 11 following the procedure described for the synthesis of compound 12a (yield 49%, over two steps). ¹H NMR (400 MHz, CDCl₃): δ 7.28 (t, J=7.2 Hz, ²H), 7.22 (d, J=7.0 Hz, ¹H), 7.19-7.13 (m, ²H), 4.63 (d, J=7.4 Hz, ¹H), 3.92 (brs, ¹H), 3.84-3.74 (m, ¹H), 3.12-2.94 (m, ³H), 2.86-2.71 (m, ²H), 2.64 (dd, J=12.1, 4.6 Hz, ¹H), 2.55 (dd, J=12.0, 7.4 Hz, ¹H), 1.71 (hept, J=6.7 Hz, ¹H), 1.58-1.21 (m, ¹³H), 0.90 (t, J=7.0 Hz, ³H), 0.86 (d, J=6.6 Hz, ⁶H).

### Synthesis of Inhibitor 19

Inhibitor 19 has been synthesized from compound 35a by Boc removal using trifluoroacetic acid followed by coupling of the resulting amine with known acid 23 using EDC, HOBt, and iPr2NEt following a similar procedure described for the synthesis of inhibitor 8 (yield 50%). ¹H NMR (400 MHz, CDCl₃): δ 7.85 (d, J=1.0 Hz, ¹H), 7.46 (s, ¹H), 7.35-7.28 (m, ²H), 7.26-7.19 (m, ³H), 6.84 (s, ¹H), 6.41 (d, J=8.2 Hz, ¹H), 4.54-4.44 (m, ³H), 3.89-3.76 (m, ³H), 3.47 (s, ³H), 3.13 (d, J=4.9 Hz, ¹H), 3.11-2.99 (m, ²H), 2.97 (d, J=6.8 Hz, ²H), 2.83 (dd, J=12.3, 4.8 Hz, ¹H), 2.79-2.68 (m, ³H), 1.71 (hept, J=6.7 Hz, ¹H), 1.54-1.33 (m, ⁴H), 1.30 (t, J=7.5 Hz, ³H), 0.91-0.80 (m, ⁹H). ¹³C NMR (100 MHz, CDCl₃): δ 172.89, 167.85, 137.51, 133.83, 130.72, 129.30, 128.67, 127.72, 127.26, 126.78, 125.83, 120.25, 117.93, 117.56, 72.00, 66.43, 56.81, 51.98, 51.20, 46.44, 43.62, 39.61, 38.94, 35.26, 28.42, 20.10, 18.98, 17.94, 14.26, 13.94. HRMS-ESI (m/z): [M+H]+ calcd for C₃₃H₄₈N₅O₅S, 626.3376; found, 626.3369.

### Synthesis of Inhibitor 20

Inhibitor 20 has been synthesized from compound 35a by Boc removal using trifluoroacetic acid followed by coupling of the resulting amine with acid 25 using EDC, HOBt, and iPr2NEt following the similar procedure described for the synthesis of inhibitor 8 (yield 52%). ¹H NMR (400 MHz, CDCl₃): δ 7.71 (d, J=0.8 Hz, ¹H), 7.40 (s, ¹H), 7.34-7.28 (m, ²H), 7.26-7.18 (m, ³H), 6.80 (s, ¹H), 6.39 (d, J=8.2 Hz, ¹H), 4.54-4.40 (m, ¹H), 4.11 (s, ²H), 3.80 (p, J=4.2 Hz, ¹H), 3.50 (s, ³H), 3.13 (d, J=4.8 Hz, ¹H), 3.11-2.99 (m, ²H), 2.97 (d, J=6.8 Hz, ²H), 2.82 (dd, J=12.3, 5.0 Hz, ¹H), 2.79-2.64 (m, ³H), 1.70 (hept, J=6.7 Hz, ¹H), 1.56 (s, ⁶H), 1.53-1.33 (m, ⁴H), 1.30 (t, J=7.5 Hz, ³H), 0.93-0.79 (m, ⁹H). ¹³C NMR (125 MHz, CDCl₃): δ 172.59, 167.99, 137.38, 134.28, 130.53, 129.22, 128.81, 128.59, 127.37, 126.83, 126.71, 120.29, 116.36, 114.86, 71.83, 66.29, 65.68, 56.50, 51.89, 51.03, 46.38, 39.40, 38.81, 35.15, 28.33, 22.49, 22.46, 20.03, 19.71, 18.90, 17.88, 14.02, 13.87. HRMS-ESI (m/z): [M+H]+ calcd for C₃₅H₅₂N₅O₅S, 654.3689; found, 654.3696.

Inhibitor 21 was synthesized (yield 65% over two steps) from 12b by Boc deprotection followed by coupling 7,6,5-tricyclic indole derivative 30 as described for the inhibitor 4. ¹H NMR (400 MHz, CDCl₃): δ 7.88 (s, ¹H), 7.47 (s, ¹H), 7.36 (t, J=6.0 Hz, ¹H), 7.33-7.27 (m, ²H), 7.26-7.17 (m, ³H), 6.77 (s, ¹H), 6.55 (d, J=8.2 Hz, ¹H), 4.56-4.43 (m, ¹H), 4.27 (s, ²H), 4.03 (p, J=6.2 Hz, ¹H), 3.41 (s, ³H), 3.12 (d, J=5.1 Hz, ¹H), 3.08-2.99 (m, ²H), 2.95 (dd, J=6.5, 4.4 Hz, ²H), 2.82 (dd, J=12.2, 4.7 Hz, ¹H), 2.78-2.65 (m, ³H), 1.69 (hept, J=6.6 Hz, ¹H), 1.52 (t, J=6.3 Hz, ²H), 1.28 (t, J=7.5 Hz, ³H), 1.13 (d, J=6.4 Hz, ³H), 1.00 (t, J=6.4 Hz, ²H), 0.83 (d, J=6.7 Hz, ³H), 0.82 (d, J=6.6 Hz, ³H). ¹³C NMR (100 MHz, CDCl₃): δ 172.75, 167.89, 137.56, 134.44, 130.80, 129.26, 128.62, 128.14, 127.09, 126.71, 126.13, 120.33, 118.15, 117.90, 68.26, 67.53, 52.30, 52.23, 51.39, 46.39, 43.05, 39.77, 39.02, 28.40, 20.09, 18.86, 17.94, 14.19, 12.67. HRMS-ESI (m/z): [M+H]+ calcd for C₃₃H₄₆N₅O₅S, 624.3220; found, 624.3223.

### Synthesis of Compound 35b

(2S,3S)-2-Amino-3-hydroxy-N-isopropylhexanamide was synthesized from (2S,3S)-2-[(tert-butoxycarbonyl)amino]-3-hydroxyhexanoic acid by coupling with isopropyl amine using EDC, HOBt, and iPr2NEt followed by boc removal using trifluoroacetic acid following the procedure described for the synthesis of (2S,3S)-2-amino-3-hydroxy-N-isobutyl-N-methyl-butanamide (yield 79% over two steps). ¹H NMR (400 MHz, CDCl₃): δ 7.22 (br, ¹H), 4.14-3.92 (m, ¹H), 3.81-3.67 (m, ¹H), 3.22 (d, J=6.2 Hz, ¹H), 2.40 (brs, ²H), 1.62-1.24 (m, ⁴H), 1.18-1.08 (m, ⁶H), 0.91 (t, J=6.9 Hz, ³H).

Compound 35b was prepared from (2S,3S)-2-amino-3-hydroxy-N-isopropylhexanamide by reductive amination with aldehyde 11 following the procedure described for the synthesis of compound 12a (yield 81%). ¹H NMR (400 MHz, CDCl₃): δ 7.28 (t, J=7.4 Hz, ²H), 7.22 (d, J=7.3 Hz, ¹H), 7.20-7.13 (m, ²H), 7.00 (br, ¹H), 4.62 (d, J=8.1 Hz, ¹H), 4.08-3.85 (m, ²H), 3.83-3.72 (m, ¹H), 2.98 (d, J=5.3 Hz, ¹H), 2.82 (dd, J=13.7, 6.5 Hz, ¹H), 2.75 (dd, J=13.3, 7.4 Hz, ¹H), 2.60 (dd, J=12.2, 4.7 Hz, ¹H), 2.54 (dd, J=12.1, 7.3 Hz, ¹H), 1.59-1.20 (m, ¹³H), 1.11 (d, J=6.5 Hz, ³H), 1.06 (d, J=6.6 Hz, ³H), 0.90 (t, J=7.0 Hz, ³H).

### Synthesis of Inhibitor 22

Inhibitor 22 was synthesized by Boc removal of 35b using trifluoroacetic acid followed by coupling of the resulting amine with the acid 30 using EDC, HOBt, and iPr2NEt following a similar procedure described for the synthesis of inhibitor 4 (yield 65% over two steps). ¹H NMR (400 MHz, CDCl₃): δ 7.88 (d, J=1.1 Hz, ¹H), 7.46 (d, J=1.0 Hz, ¹H), 7.36-7.28 (m, ²H), 7.28-7.20 (m, ³H), 6.97 (d, J=8.2 Hz, ¹H), 6.78 (s, ¹H), 6.38 (d, J=8.2 Hz, ¹H), 4.54-4.42 (m, ¹H), 4.28 (ABq, J=20.6, 14.9 Hz, ²H), 4.08-3.95 (m, ¹H), 3.84-3.75 (m, ¹H), 3.44 (s, ³H), 3.09 (d, J=4.9 Hz, ¹H), 2.98 (d, J=6.8 Hz, ²H), 2.81 (dd, J=12.3, 4.9 Hz, ¹H), 2.77-2.69 (m, ³H), 1.56-1.33 (m, ⁵H), 1.33-1.24 (m, ⁴H), 1.07 (t, J=6.9 Hz, ⁶H), 1.01-0.96 (m, ²H), 0.87 (t, J=7.1 Hz, ³H). ¹³C NMR (125 MHz, CDCl₃): δ 171.67, 167.77, 137.47, 134.36, 130.68, 129.20, 128.58, 128.06, 126.97, 126.69, 126.05, 120.23, 118.05, 117.83, 71.81, 66.05, 52.15, 51.63, 50.99, 43.01, 40.92, 39.60, 38.81, 35.05, 22.46, 18.90, 17.89, 14.15, 13.86, 12.64. HRMS-ESI (m/z): [M+H]+ calcd for C₃₄H₄₈N₅O₅S, 638.3376; found, 638.3371.

### Determination of X-Ray Structure of β-Secretase-Inhibitor 5 Complex

Expression and purification of recombinant human β-secretase, crystal growing, inhibitor soaking of the crystal, and diffraction data collection were as previously described.8 The structure was determined by molecular replacement implemented with the program AMoRe using the C molecule of previously determined memapsin 2 structure (PDB ID: 1FKN) as a search model8 with removed inhibitor and water molecules. Rotation and translation functions followed by the rigid-body refinement with data from 15 to 3.5 Å resolution in space group P21 gave unambiguous solutions for the four memapsin 2 molecules in the asymmetric unit. A random selection of 7% of reflections (9028 reflections) was set aside as the test set for cross-validation during the refinement. The refined model had well-defined electron density for the inhibitor, and its corresponding structure was built into the active site. The four molecules in the crystallographic asymmetric unit have essentially identical structures. The crystal form was determined to be monoclinic with a resolution of 2.0 Å. The unit cell parameters are a=86.4 Å, b=130.3 Å, c=88.4 Å, and β=97.5. The coordinates and structure factors of the β-secretase and 5 complex have been deposited in Protein Data Bank28 with PDB ID: 4GID.

### Example 2

### Results

A reduced amide β-secretase inhibitor 4 was synthesized by the inventors, and this compound has exhibited a BACE 1 Ki of 27 nM and marginal selectivity against BACE 2 and CD in in-house enzyme inhibitory assays. An energy-minimized model of 4 was created based upon the protein-ligand X-ray structure of 2-bound β-secretase.9 The preliminary model suggested that an introduction of a hydroxyl group with S-configuration on the ethyl group of homoalanine moiety would make enhanced interactions in the active site and possibly enhance selectivity. On the basis of this molecular insight, the inventors have designed, synthesized, and evaluated inhibitors 5 and 6 (FIG. 2) to investigate the influence of the hydroxyl group and also the role of stereochemistry on the potency and selectivity. They have also synthesized and evaluated inhibitors 7-9 to examine the importance of various functional groups at the P1 97′ and P2′ sites on the potency.

The BACE 1 inhibitory activity of synthetic inhibitors 4-9 was determined against recombinant β-secretase using previously reported assay protocols (Ermolieff et al., 2000). The results are shown in Table 1. As can be seen, inhibitor 4 with a homoalanine P1′ side chain has shown a Ki of 27 nM (entry 1). Inhibitor 5 with an allothreonine P1′ side chain has exhibited remarkable BACE 1 inhibitory activity with a Ki of 17 pM (entry 2). Inhibitor 6 with a threonine P1′ side chain has shown significant reduction of BACE 1 activity over the deoxyderivative 4 or the inhibitor 5 with an allothreonine P1′ side chain (entries 1-3). The inventors have also evaluated the cellular inhibition of β-secretase in neuroblastoma cells. (Chang et al., 2004). Consistent with potent BACE 1 inhibitory activity, inhibitor 5 exhibited an average cellular IC₅₀ value of 1 nM. The corresponding inhibitor 6 with a threonine P1′ side chain has shown a cellular IC50 value of >1 μM in the same assay. Inhibitor 7 with an N-methyl amide abolished all BACE 1 inhibitory activity. Inhibitor 8 with an “OMe” group in the P1′ region also showed substantial reduction in inhibitory potency (entry 5) as compared to inhibitor 5 with hydroxyl group. Inhibitor 9 with a reduced amide in the P2′ region resulted in a total loss in potency. These results clearly demonstrate the significance of hydrogen-bonding interactions of inhibitor 5 with the prime region of the BACE 1 active site.

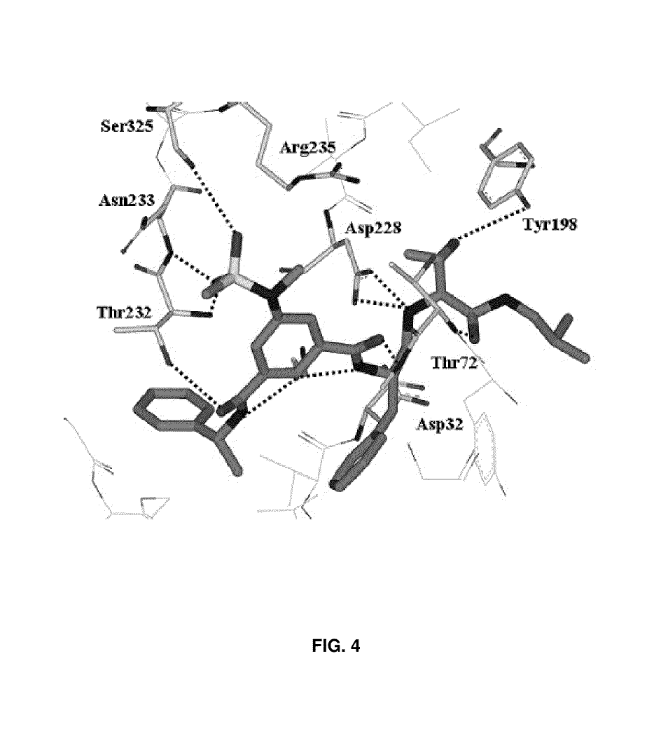

To gain further molecular insight, the inventors have determined the X-ray structure of 5-bond to β-secretase at a 2.2 Å resolution. As shown in FIG. 4, the amine functionality of the reduced amide isostere forms two tight hydrogen bonds (2.4 and 2.7 Å bond distances) with active site aspartic acid Asp228 (Ghosh et al., 2000). Interestingly, the other active site Asp32 is not directly interacting with the reduced amide isostere. Asp32 is extensively hydrogen bonded to three groups: the amide nitrogen of Gly34 (2.8 Å), the hydroxyl group of Ser35 (2.5-3 Å with rotations of the involved groups), and the amide nitrogen of Gly230 (3.3 Å). These interactions appear to lock Asp32 in a rigid conformation; thus, its hydrogen bonding to Asp228 produced a network of hydrogen bond interactions to include the interaction of the inhibitor and the protease. A similar inhibitor-enzyme interacting pattern has been reported for the crystal structure of reduced amide isosteres (Coburn et al., 2006). The fact that inhibitors 2 and 5 are highly potent suggests that the interaction of both active site carboxyls with the transition-state isostere is not a necessary feature for the design of potent inhibitors. The P3-phenyl ring occupies a unique position that spans S3 and S4 subsites and causes a significant positional shift of a protein loop containing residues from 8 to 13 (the 10 s loop) (Patel et al., 2004) located in the S3/S4 pocket similar to inhibitor 2. This flexible part of the active site cleft can be further exploited for ligand design. The P2 240′-carbonyl as well as P2′-NH are within proximity to form hydrogen bonds with Thr72 and Gly34, respectively. Most significantly, the allothreonine hydroxyl group is oriented toward the Tyr-198 hydroxyl group. This interaction is presumably absent in inhibitor 4. Also, the P1′-hydroxyl group stereochemistry is optimal for critical hydrogen bonding with Tyr-198. The combinations of active site interactions are responsible for the potency and selectivity of inhibitor 5.

The X-ray crystal structure of 5-bound memapsin 2 demonstrates the importance of the allothreonine moiety as it forms key hydrogen-bonding interactions with the prime region of memapsin 2. Therefore, inhibitors 18-22, with a 7,6,5-tricyclicindole moiety as the P2 ligand, were designed with a view to reduce labile amide bonds in isophthalic acid amide-derived ligand. Synthetic inhibitors 18-22 were evaluated against recombinant BACE 1, and the results are summarized in Table 2. Inhibitor 18, containing a known 7,6,5-tricyclic moiety as the P2 ligand, showed a low nanomolar activity toward BACE 1 (Ki=7.3 nM). This inhibitor is substantially less potent than inhibitor 5; however, the ratio of cell inhibitory to enzyme inhibitory efficacy was improved significantly (3 vs >58), indicating better cell permeability for compound 18. Inhibitor 19 with a sterically more demanding propyl group in the P1′ region has shown around 18-fold improvement in the potency (entries 1 and 2). Inhibitor 20 with a dimethyl-substituted indole derivative as the P2 ligand resulted in >10-fold potency enhancement over unsubstituted inhibitor 19. This inhibitor exhibited a cellular IC₅₀ value of 15 nM. The ligand was also designed especially to halt the possibility of retro-Michael reaction of the P2-α,α-unsubstituted sultam functionality in inhibitors 18 and 19 (Charrier et al., 2009). Inhibitors 21 and 22, designed by replacing the two methyl groups of P2 ligand in 20 with cyclopropyl group, also exhibited impressive potency.

We then evaluated the potencies of selected inhibitors against recombinant BACE 2 and human CD, and the results are shown in Table 3. Interestingly, inhibitor 5 displayed very impressive selectivity against BACE 2 (Ki=120 nM, selectivity>7000-fold) and CD (Ki=4.3 μM, selectivity>250000-fold) as well. In comparison, deoxyinhibitor 4 has shown a BACE 2 Ki of 1450 nM (selectivity>50-fold) and CD Ki of 8264 nM (selectivity>300-fold). This result suggested that the allothreonine hydroxyl group on the P1′ side chain is critical to the selectivity and potency of inhibitor 5. Inhibitor 18 has also exhibited good selectivity (over 970-fold selective) toward BACE 1 over CD (entry 3). Inhibitor 19 with a butyl side chain has shown improvement in CD selectivity (entry 4). Inhibitor 20 displayed good selectivity against BACE 2 and excellent selectivity against CD (entry 5). Inhibitor 22 has also shown a >4200-fold selectivity over CD (entry 6).

### Example 3

### Discussion

In conclusion, the inventors have designed, synthesized, and examined the biological activity of isophthalamide-based BACE 1 inhibitors containing various functional groups in the prime region. The selectivity of inhibitors 4 and 5 against BACE 2 and CD was also examined. Inhibitor 5 with an allothreonine moiety exhibited superior potency and exceptionally high selectivity when compared to inhibitors with a threonine moiety or an ethylglycine (homoalanine) moiety. Inhibitors with N-isobutyl-N-methylamide and P2′ reduced amide groups on the prime side lost their efficacy. These results clearly demonstrate the significance of prime region of the inhibitors on the potency and selectivity. The X-ray structure of 5-bond β-secretase also showed the presence of an effective hydrogen bond between the prime side of the inhibitor and Thr72, Gly34, and Tyr 198 residues of BACE 1. On the basis of this molecular insight, the inventors have further designed and synthesized inhibitors by replacing the isophthalamide moiety with conformationally constrained 7,6,5-tricyclicindole moieties and by keeping the allothreonine moiety intact. These inhibitors have also shown very good potency and selectivity against CD. These results further support the significance of hydrogen-bonding interactions in the prime region for the potency and selectivity. The combination of active site interactions along with the Tyr-198 may be responsible for the observed selectivity. This molecular insight may aid further design of selectivity against other aspartic acid proteases. Further investigations into the origin of selectivity are in progress.

TABLE 1
BACE 1 Inhibitory and Cellular Activity of Inhibitors 4-9

| Entry | Inhibitor | Kᵢ (nM) | IC₅₀ (nM)^{a} |
|---|---|---|---|
| 1 | 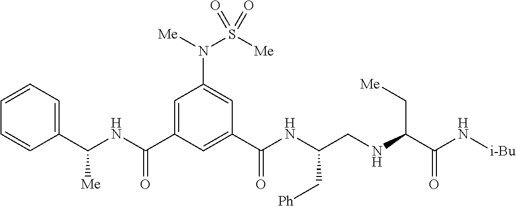 4 | 27.12 | 9.5 |
| 2 | 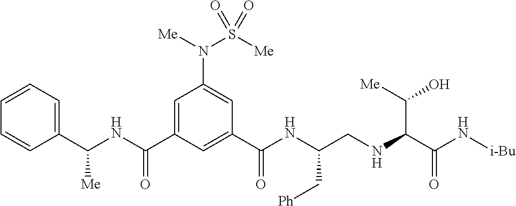 5 | 0.017 | 1 |
| 3. | 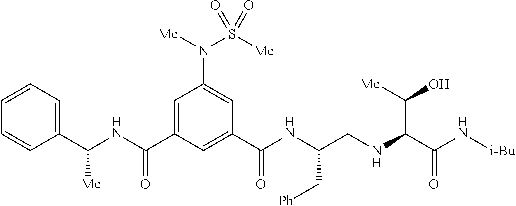 6 | 98.8 | >1000 |
| 4. | 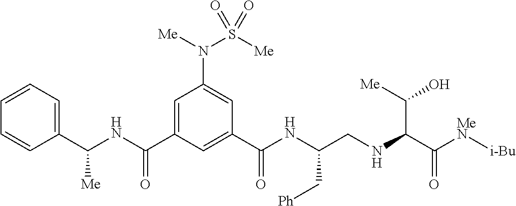 7 | >1000 | — |
| 5. | 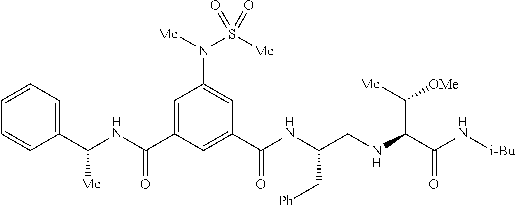 8 | 25 | — |
| 6. | 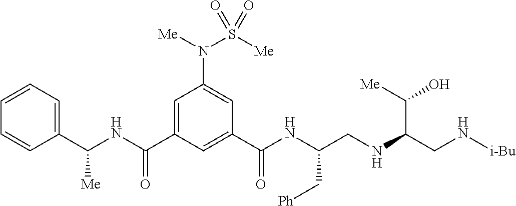 9 | >1000 | — |

^{a}The IC₅₀ was determined in neuroblastoma cells. GRL-8234 exhibited Kᵢ = 1.8 nM and IC₅₀ = 2.5 nM in this assay.

TABLE 2
BACE 1 Inhibitory and Cellular Activity of
Inhibitors 18-22

| Entry | Inhibitor | Kᵢ (nM) | IC₅₀ (nM)^{a} |
|---|---|---|---|
| 1 | 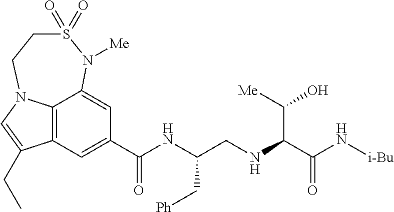 18 | 7.3 | 22 |
| 2 | 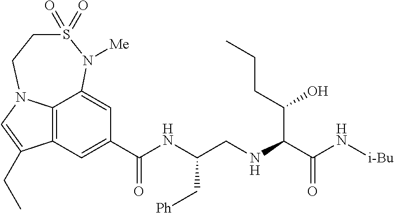 19 | 0.4 | — |
| 3 | 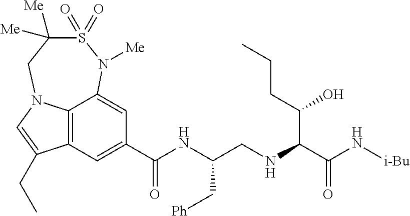 20 | 0.036 | 15 |
| 4 | 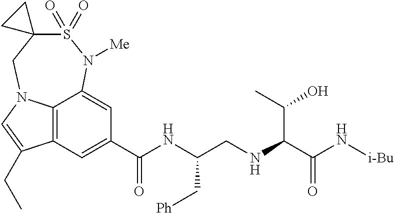 21 | 7.2 | — |
| 5 | 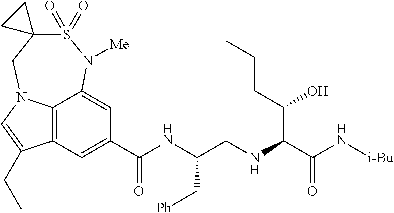 22 | 0.47 | 14 |

^{a}The IC₅₀ was determined in neuroblastoma cells. GRL-8234 exhibited Kᵢ = 1.8 nM and IC₅₀ = 2.5 nM in this assay.

TABLE 3
Selectivity Studies of BACE 1 Inhibitors against BACE 2 and CD

| | | | | | selectivity | |
|---|---|---|---|---|---|---|
| | | Kᵢ (nM) | | | BACE 2/ | CD/ |
| entry | inhibitor | BACE 1 | BACE 2 | CD | BACE 1 | BACE 1 |
| 1 | 4 | 27.12 | 1450 | 8264 | >55 | >300 |
| 2 | 5 | 0.017 | 120 | 4300 | >7000 | >250000 |
| 3 | 18 | 7.3 | | 7100 | | >970 |
| 4 | 19 | 0.4 | | 690 | | >1700 |
| 5 | 20 | 0.036 | 11 | 530 | >300 | >14700 |
| 6 | 22 | 0.47 | | 2000 | | >4200 |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other details supplementary to those set forth herein, are specifically incorporated herein by reference.
Billings et al., Neuron 45:675-688, 2005.
Chang et al., FASEB J. 25:775-784, 2011.
Chang et al., J. Neurochem. 89:1409-1416, 2004.
Charrier et al., Bioorg. Med. Chem. Lett. 19:3674-3678, 2009.
Charrier et al., J. Med. Chem. 51:3313-3317, 2008.
Charrier et al., Synthesis 3467-3477, 2006.
Chartier-Harlin et al. Nature 353:844-846, 1991.
Citron et al., Nature 360:622-674, 1992.
Citron, Nat. Rev. Drug Discovery 9:387-398, 2010.
Coburn et al., Bioorg. Med. Chem. Lett. 16:3635-3638, 2006.
Corder, Science 261:921-923, 1993.
Davies, D. R., Annu. Rev. Biophys. Chem. 19, 189, 1990.
Diment et al., J. Biol. Chem. 263:6901-6907, 1988.
Ermolieff et al., Biochemistry 39:12450-12456, 2000.
Fleming and Sharpless, J. Org. Chem. 56:2869-2875, 1991.
Ghosh et al., Bioorg. Med. Chem. Lett. 18:1031-1036, 2008.
Ghosh et al., J. Am. Chem. Soc. 122:3522-3523, 2000.
Ghosh et al., J. Am. Chem. Soc. 128:5310-5311, 2006.
Ghosh et al., J. Neurochem. 120:71-83, 2012.
Glenner and Wong, Biochem. Biophys. Res. Comm. 120:885-890, 1984.
Goate et al. Nature 349:704-706, 1991.
Goedert and Spillantini, Science 314:777-781, 2006.
Goldgaber et al., Science 235:877-880, 1987.
Hong et al., Science 290:150-153, 2000.
Hubert et al, Tricyclic Indole Derivatives and Their Use in the Treatment of Alzheimer's Disease, 2004.
Iserloh and Cumming, In Aspartic Acid Proteases as Therapeutic Targets; Ghosh, A. K.,
Ed.; Wiley-VCH: Weinheim, Vol. 45, pp 441-479, 2010.
Kang et al., Nature 325:733-735, 1987.
Kelly, Microbiol. Sci. 1(9):214-219, 1984.
Koike et al., J. Neurosci. 20:6898-6906, 2000.
Labby et al., Bioorg. Med. Chem. 20:2435-2443, 2012.
Lemaire et al., Nucl. Acids Res. 17:517-522, 1989.
Levy et al., Science 248:1124-1128, 1990.
Levy-Lahad et al., Science 269:973-977, 1995.
Mann et al., Neurobiol. Aging 10:397-399, 1989.
Mullan et al., Nature Genet. 1:345-347, 1992.
Murrell et al. Science 254:97-99, 1991.
Patel et al., J. Mol. Biol. 343:407-416, 2004.
Podlisny et al., Neurobiol. Dis. 3:325-37, 1997.
Robakis et al., Proc. Natl. Acad. Sci. 84:4190-4194, 1987.
Rogaev et al., Nature 376:775-778, 1995.
Rumble et al., New England J. Med. 320:1446-1452, 1989.
Saftig et al., K. EMBO J. 14:3599-3608, 1995.
Selkoe and Schenk, Annu. Rev. Pharmacol. Toxicol. 43:545-584, 2003.
Selkoe, D. J. Nature 399:23-31, 1999.
Sherrington et al., Nature 375:754-760, 1995.
Sinha & Lieberburg, Proc. Natl. Acad. Sci., USA, 96:11049-11053, 1999.
Suzuki et al., Science 264:1336-1340, 1994.
Tang et al., In Aspartic Acid Proteases as Therapeutic Targets; Ghosh, A. K., Ed.; Wiley-VCH: Weinheim, Vol. 45, pp 413-440, 2010.
Tanzi et al., Nature 331:528-530, 1988.
Thinakaran et al., Neuron 17:181-190, 1996.
Turner et al., Biochemistry 41:8742-8746, 2002.
Van Broeckhaven et al., Science 248:1120-1122, 1990.
Wolfe et al., Nature 398:513-517, 1999.
Yoshikai et al., Gene 87, 257-263, 1990.

1. A method of inhibiting memapsin 2 activity comprising contacting a memapsin 2 enzyme with a compound having a formula selected from: 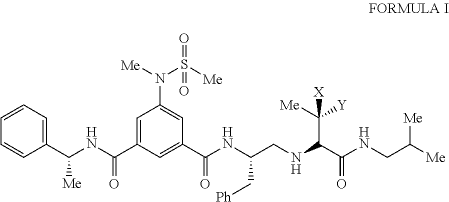
wherein X and Y are H or OH; or 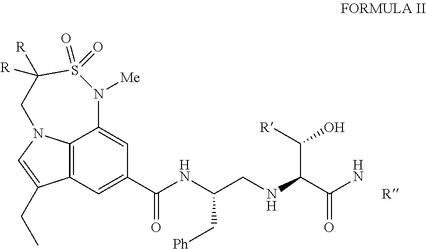
wherein each R, R′ and R′ are independently selected from C_{<8} alkyl, C_{<8} substituted alkyl, C_{<8} heterocycloalkyl, C_{<8} alkoxyalkyl, C_{<9} alkylamino, C_{<12} aryl, C_{<12} arylalkyl,
or a pharmaceutically acceptable salt or tautomer of any of the above formulas.

2-10. (canceled)

11. A method of treating a mammalian subject with Alzheimer's Disease comprising administering to said subject a compound having a formula selected from: 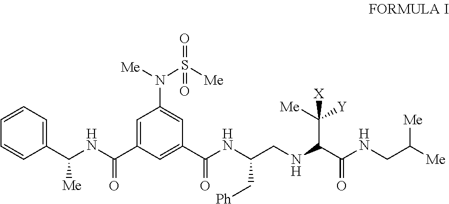
wherein X and Y are H or OH; or 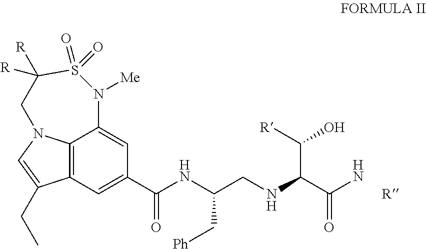
wherein each R, R′ and R′ are independently selected from C_{<8} alkyl, C_{<8} substituted alkyl, C_{<8} heterocycloalkyl, C_{<8} alkoxyalkyl, C_{<9} alkylamino, C_{<12} aryl, C_{<12} arylalkyl,
or a pharmaceutically acceptable salt or tautomer of any of the above formulas.

12-20. (canceled)

21. The method of claim 11, wherein said subject is further treated with at least a second Alzheimer's Disease therapy.

22. (canceled)

23. The method of claim 11, wherein treating comprises one or more of improvements in memory, cognition or learning, slowing the progression of symptoms or pathophysiology, improving quality of life, or increasing life span.

24. The method of claim 11, wherein said compound is administered orally or by injection, including intravenously, intradermally, intraarterially, intraperitoneally, intracranially, intraarticularly, intraprostaticaly, intrapleurally, intramuscularly, or subcutaneously.

25. (canceled)

26. The method of claim 11, further comprising measuring cognition or memory in said subject prior to and/or after administration of said compound.

27. The method of claim 11, wherein said mammalian subject is a human.

28. A compound having a formula selected from: 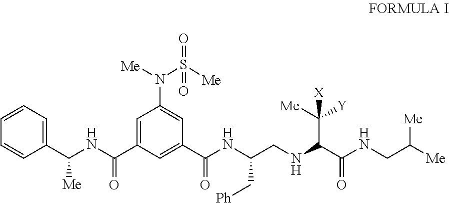
wherein X and Y are H or OH; or 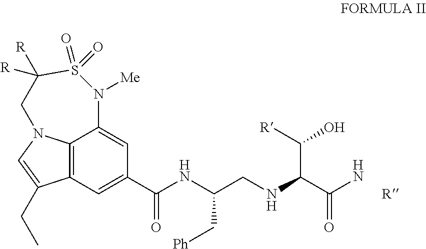
wherein each R, R′ and R′ are independently selected from C_{<8} alkyl, C_{<8} substituted alkyl, C_{<8} heterocycloalkyl, C_{<8} alkoxyalkyl, C_{<9} alkylamino, C_{<12} aryl, C_{<12} arylalkyl,
or a pharmaceutically acceptable salt or tautomer of any of the above formulas.

29. The compound of claim 28, wherein the compound has formula I, wherein X is H and Y is OH.

30. The compound of claim 28, wherein the compound has formula I, wherein X is H and Y is H.

31. The compound of claim 28, wherein the compound has formula II, wherein R is H, R′ is —CH₃, and R″ is isobutyl.

32. The compound of claim 28, wherein the compound has formula II, wherein R is H, R′ is n-propyl, and R″ is isobutyl.

33. The compound of claim 28, wherein the compound has formula II, wherein R is H, R′ is isopropyl, and R″ is isobutyl.

34. The compound of claim 28, wherein the compound has formula II, wherein each R together form —CH₂—CH₂—, R′ is —CH₃, and R″ is isobutyl.

35. The compound of claim 28, wherein the compound has formula II, wherein each R together form —CH₂—CH₂—, R′ is n-propyl, and R″ is isobutyl.

36. The compound of claim 28, wherein the compound has formula II, wherein each R together form —CH₂—CH₂—, R′ is isopropyl, and R″ is isobutyl.

37. The compound of claim 28, wherein the compound has formula II, wherein R′ is isopropyl.

38. A pharmaceutical composition comprising a compound according to claim 28 formulated in a pharmaceutical buffer, diluent or excipient.

39. The pharmaceutical composition of claim 38, wherein said composition is in a solid dosage form such as a tablet, a capsule or a powder.

40. The pharmaceutical composition of claim 38, wherein said composition is in a liquid dosage form.
